(19) United States
(12) Patent Application Publication
(10) Pub. No.: US 2009/0252769 A1
(43) Pub. Date: Oct. 8, 2009

(54) PREVENTIVE AND THERAPEUTIC VACCINE FOR STORKE AND NEUROLOGICAL DISORDERS

(76) Inventor: Jeeri R Reddy, Omaha, NE (US)

Correspondence Address: JEERI R. REDDY, 6912 NORTH 97TH CIRCLE, SUITE C, OMAHA, NE, 68122, US

(21) Appl. No.: 12/307,587

(22) PCT Filed: Jun. 6, 2007
(86) PCT No.: PCT/US07/70542

§ 371 Date: Jan. 6, 2009

(30) Foreign Application Priority Data
Jun. 6, 2007 (US) PCT/US2007/070542

(51) Int. Cl. A61K 9/00; A61K 39/00

(52) U.S. Cl. 424/400; 424/185.1

(57) ABSTRACT

A method for producing therapeutic vaccine which consist of NMDA-NR1 subunit expressed in insect cells to produce recombinant protein and was encapsulated in poly(D-L-lactide-co-glycolic-acid) (PGLA) microparticles by solvent exchange and used for oral immunization. Thus the experimental model for stroke has been developed for the study of powerful N-methyl-d-aspartic acid (NMDA) NR1 sub units, their protective and therapeutic potential for treatment of the neurological disorder of stroke in animals and its practicability for therapy in humans.

### CROSS REFERENCE TO RELATED APPLICATIONS

This application is based on provisional application U.S. Ser. No. 60/909,449 filed on Mar. 31, 2007.

### BACKGROUND OF THE INVENTION

1. Field of the Invention

The present invention is related generally to the field of methods and compositions of prevention and treatment of neurological disorders, such as epilepsy and stroke, Alzheimer's, Parkinson's, dementia, Huntington's disease, amyloid lateral sclerosis and depression, and neuroendocrine disorders such as obesity.

2. Description of the Related Art

NMDA (N-methyl-D-aspartic acid) is an amino acid derivative which acts as a specific agonist of the NMDA receptor because it mimics the action of the neurotransmitter glutamate on that receptor. In contrast to glutamate, NMDA binds to and regulates only the NMDA receptors, and does not affect other glutamate receptors. NMDA is a water-soluble synthetic substance that is not normally found in biological tissue. At physiological pH both of the carboxyl groups of NMDA are deprotonated. NMDA is used as an excitotoxin in behavioral neuroscience research and studies utilizing this technique fall under the term “lesion studies.”

Activation of NMDA receptors allows both sodium and calcium ions to enter the postsynaptic neuron which results in long-term changes in the postsynaptic membrane that make it more sensitive to synaptic input. This long-term potentiating effect may be a rudimentary component of memory. Since the NMDA receptor (NMDAR) is one of the most harmful factors involved in excitotoxicity, it is a logical target for the treatment of excitotoxicity. Excitotoxicity is involved in a number of neurological conditions including traumatic brain injury, stroke, and neurodegenerative diseases such as Alzheimer, Parkinson, and Huntington. NMDAR antagonists have great potential utility for the treatment of conditions that involve excitotoxicity. However, because of the neurotoxicity caused by NMDAR antagonists, research has been focused on finding agents that avoid this neurotoxicity. Most clinical trials involving NMDAR antagonists have failed because of unwanted side effects of the drugs. Since these receptors play an important role in normal glutamatergic function, blocking them can have potentially harmful effects. This interference with normal function could be responsible for the neuronal death that sometimes results from NMDAR antagonist use. In addition, inadequate NMDAR function is associated with an array of negative symptoms. For example, NMDAR hypofunction that occurs as the brain ages may be partially responsible for memory deficits.

NMDA Receptor (NMDAR): NMDA is the selective, specific agonist of the NMDAR, an ionotropic ligand-gated and voltage-dependent receptor for glutamate. Activation of NMDARs results in the opening of a nonselective cation channel which allows the flow of Na+ and K+ ions, and small amounts of Ca2+ ions. The resultant calcium-flux through NMDARs is thought to play a critical role in synaptic plasticity providing a cellular mechanism for learning and memory.

The recently solved structure of NMDAR at atomic resolution has revealed that there is a heterodimer formed between the NR1 and NR2 subunits. This structural analysis explains why NMDARs contain two obligatory NR1 subunits and two regionally localized NR2 subunits. A related gene family of NR3A and B subunits has an inhibitory effect on NMDAR activity. Multiple receptor isoforms, each with distinct distributions within the brain and with distinct functional properties, arise by the selective splicing of the NR1 transcripts and the differential expression of the NR2 subunits.

Each receptor subunit has a modular design with each structural module representing a functional unit. Two globular structures, a modulator domain and a ligand-binding domain, are found in the extracellular portion of the subunit. The NR1 subunits bind the co-agonist glycine while the NR2 subunits bind the neurotransmitter glutamate. The solution of the three-dimensional structure of the glycine-binding module of the NR1 subunit and the glutamate-binding module of the NR2A subunit, by X-ray crystallography, revealed a common fold with the glutamate-binding module of AMPA-receptors and kainate-receptors. The agonist-binding module is linked to a membrane domain consisting of three trans-membrane segments and a re-entrant loop reminiscent of the selectivity filter of potassium channels. The membrane domain contributes residues to the channel pore and is responsible for the receptor's high unitary conductance, high calcium permeability and the voltage-dependent magnesium block. Each subunit also has an extensive cytoplasmic domain which contains residues that are directly modified by a series of protein kinases and protein phosphatases, as well as residues which interact with a large number of structural, adaptor and scaffolding proteins.

Agonists: Activation of NMDARs requires the binding of both glutamate and the co-agonist glycine for the efficient opening of the ion channel which is a part of this receptor. D-serine has also been found to act as a co-agonist of the NMDAR with even greater potency than glycine. D-serine is produced by serine racemase in astrocyte cells and is enriched in the same areas as NMDARs. Removal of D-serine can block NMDA-mediated excitatory neurotransmission in many areas.

In addition, a third requirement for activation is membrane depolarization. A positive change in trans-membrane potential will make it more likely that the ion channel in the NMDAR will open by expelling the Mg2+ ion that blocks the channel from the outside. The latter function is fundamental to the role of NMDAR for both memory and learning, perhaps by acting as a coincidence detector for membrane depolarization and synaptic transmission.

Antagonists: The NMDAR channel complex contributes to excitatory synaptic transmission at sites throughout the brain and the spinal cord, and is modulated by a number of endogenous and exogenous compounds. NMDARs play a key role in a wide range of physiologic and pathologic processes.

NMDAR antagonists are a class of anesthetics that work to antagonize, or inhibit the action of, NMDAR. They are used as anesthesia for animals and (less commonly) for humans, and some, such as ketamine and phencyclidine (PCP) are also popular as recreational drugs because of their hallucinogenic properties. When used recreationally they are classified as dissociative drugs, and are often considered entheogens. When NMDAR antagonists are given to rodents in large doses, they can cause a form of brain damage called Olney's Lesions. However, there is insufficient research to show that large doses of NMDAR antagonists cause Olney's Lesions in humans and there are known to be fundamental differences between human and rodent brains. (Farber, et al., 2003. Muscimol prevents NMDA antagonist neurotoxicity by activating GABAA receptors in several brain regions. Brain Res. 993: 90-100).

Autoantibodies that targeted a specific brain protein, the NR1 subunit of NMDAR, were generated after treatment with an adeno-associated virus (AAV) vaccine carrying a recombinant NMDA-NR1. Following oral administration of the NMDA-NR1 DNA in mice there was a robust humoral response in the absence of a significant cell mediated response. This single dose vaccine provided a strong anti-epileptic and neuroprotective activity in mice for both a kainate-induced seizure model and an endothelian-1 (ET-1) induced middle cerebral artery occlusion (MCAO) stroke model at 1 to 5 months following vaccination. Thus, a vaccination strategy targeting brain proteins is feasible and may have therapeutic potential for treatment of neurological disorders (During et al., 2000. An oral vaccine against NMDAR1 with efficacy in experimental stroke and epilepsy. Science 287:1453-1460).

The NMDAR is modulated by a number of endogenous and exogenous compounds. Mg2+ blocks the NMDAR channel in a voltage-dependent manner, but also potentiates NMDA-induced responses at positive membrane potentials. Magnesium treatment has been used to produce rapid recovery from depression. Na+, K+ and Ca2+ not only pass through the NMDAR channel but also modulate the activity of NMDARs. Zn2+ blocks the NMDA current in a noncompetitive and a voltage-independent manner. Polyamines do not directly activate NMDA receptors, but have been found to potentiate or inhibit glutamate-mediated responses. The activity of NMDARs is also extremely sensitive to changes in H+ concentration, and is partially inhibited by the ambient concentration of H+ under physiological conditions.

Role of NMDA Receptors: Both the NMDA and non-NMDA subclasses of glutamate receptors are mediators of glutamatergic excitatory neurotransmission in the central nervous system (CNS). NMDARs are of particular interest because they are involved in many processes which are necessary during the development of the brain, including neuronal migration, patterning of afferent termination, and several forms of long-term synaptic plasticity. Relevant properties of the receptor include calcium permeability, voltage dependent Mg2+ block, and slow channel kinetics. Three receptor subunit families (NR1, NR2, and NR3A), which form hetero-oligomeric complexes in native NMDAR channels have been identified by molecular cloning. The formation of functional NMDARs requires the involvement of the NR1 subunits, whereas the other subunits modify the properties of the receptor. In addition to the role of NMDARs in brain plasticity and development, they have been implicated as mediators of neuronal injury associated with many neurological disorders including stroke, epilepsy, brain trauma, dementia, and neurodegenerative disorders.

Pharmacological NMDAR antagonists have been evaluated for potential clinical use because of the central involvement of NMDARs in the cascade leading to neuronal death following a variety of cerebral insults. Although these drugs are effective in many experimental animal models of diseases, the initial enthusiasm for this approach has waned because the therapeutic ratio for most NMDAR antagonists is poor, with significant adverse effects at clinically effective doses, which has limited their utility.

We investigated the hypothesis that a humoral autoimmune response, targeting the NR1 subunit of NMDAR, could be used as an alternative approach to antagonize NMDARs and provide neuroprotection. We also hypothesized that such autoantibodies would have minimal penetration into the CNS under basal conditions and would thereby avoid the toxicity associated with traditional approaches. Following cerebral insult, these autoantibodies would pass into the brain more efficiently, antagonize the receptor, and thereby attenuate NMDAR mediated injury.

Bioadhesive microspheres in the Targeted Vaccine Delivery System: The term “bioadhesion” describes materials that bind to biological substrates, such as mucosal membranes. Bioadhesive microspheres exhibit a prolonged residence time at the site of application or absorption and facilitate an intimate contact with the underlying absorption surface, contributing to the improved therapeutic performance of vaccines. The use of bioadhesive microspheres as vaccine delivery devices to mucosal tissues offers the possibility of creating a prolonged, intimate contact at the site of administration. Prolonged residence time can result in enhanced absorption, which in combination with a controlled release of the vaccine can improve patient compliance by reducing the frequency of administration. This approach to the development of a vaccine delivery system involves coupling the vaccine to a carrier particle such as microspheres or nanospheres which can modulate the release and mucosal absorption of the vaccine by virtue of their small size and efficient carrier capacity. The success of these microspheres is limited because they possess a short residence time at the site of absorption. Intimate contact of the vaccine delivery system with the absorbing membranes can be achieved by developing bioadhesive microspheres. Bioadhesive microspheres include microparticles and microcapsules, containing a core of vaccine, which consist entirely of a bioadhesive polymer or have an outer coating of polymer. Microspheres have the potential to be used for targeted and controlled-release vaccine delivery; but coupling of bioadhesive properties to microspheres provides additional advantages. These include efficient absorption and enhanced bioavailability of the vaccines because of a high surface to volume ratio, resulting in a much more intimate contact with the mucus layer. Specific targeting of vaccines to the absorption site is achieved by adding anchoring sites to the surface of the microspheres.

Bioadhesive microspheres can be prepared using different techniques and can be tailored to adhere to any mucosal tissue including those found in eye, nasal cavity, urinary tract, colon and gastrointestinal tract, offering the possibilities of localized as well as systemic controlled release of vaccines. Application of bioadhesive microspheres to specific mucosal tissues can also be used for localized vaccine action. Prolonged release of vaccines leading to reduction in frequency of vaccine administration can greatly improve patient compliance. Microspheres prepared with bioadhesive and bioerodible polymers undergo selective uptake by the M cells of Peyer patches in gastrointestinal (GI) mucosa. This uptake mechanism has been used for the delivery of protein and peptide antigens for vaccination and plasmid DNA for gene therapy.

Mucosal Immune System: Constant stimulation of the immune system is provided by diverse environmental antigens derived from ingested food, inhaled and ingested microorganisms and endogenous bacterial flora, which come into contact with mucosal tissues. The mucosal tissues and secretory glands comprise the largest accumulation of T cells, B cells, and plasma cells in the body, as well as a full complement of antigen-presenting cells which are able to participate in the initiation of specific B and T cell-mediated immune responses, far exceeding the numbers of such cells found in the bone marrow, spleen and lymph nodes. The overwhelming proportion of antibodies is produced locally in mucosal tissues and in most species, including humans, antibodies derived from the circulation represent only a minor fraction.

It is important that innovative approaches be used in further studies to critically evaluate the role of the nasopharyngeal lymphoid tissues in human mucosal immunity. Although most investigations on IgA inductive sites have centered on Peyer patches (PP) and the appendix, analogous follicular structures also occur in the large intestine, especially in the rectum. Several studies have suggested the potential importance of the rectal lymphoid tissues as a site of induction of IgA and as a source of IgA plasma cell precursors which are destined for the genital tract. In humans, there is an unusual predominance of IgA2 plasma cells in the lamina propria of the large intestine and in the female genital mucosal tissues (uterus, cervix, fallopian tubes, and vagina) (Crago et al., 1984. Distribution of IgA1-, IgA2-, and J chain-containing cells in human tissues. J. Immunol. 132:16-18 and Mestecky and Russell 1986. IgA subclasses. Monogr. Allergy. 19:277-301). Intrarectal or intranasal immunization may be particularly effective in generating antibodies in the female genital tract. These studies suggest that sub-compartmentalization may exist within the context of the common mucosal immune system which can be controlled by the homing preference of IgA plasma cell precursors, such that certain IgA-inductive sites may preferentially provide precursor lymphocytes for a particular effector site. The design of effective vaccines could be facilitated by further comparative studies of the distribution of specific secretory IgA (S-IgA) antibodies induced by diverse mucosal immunization routes.

Delivery of DNA to Mucosal Surfaces: An alternative approach for mucosal vaccine delivery is the direct administration to mucosal surfaces of a plasmid DNA expression vector which encodes the gene for a specific protein antigen. Preparation of the plasmid DNA is both simple and inexpensive. Plasmid vectors are more efficient for gene transfer to muscle tissue. The potential to deliver DNA vectors to mucosal surfaces by oral administration has been reported (PLGA encapsulated Rotavirus and Hepatitis B) and DNA plasmids have been utilized for direct introduction of genes into other tissues. DNA vaccines have been introduced into animals primarily by intramuscular injection or by gene gun delivery. After being introduced, the plasmids are maintained episomally without replication. Expression of the encoded proteins has been shown to persist for extended time periods, providing constant stimulation of B and T cells. Administration of vaccine plasmid DNA introduces the antigen directly into the pathway that results in the generation of cell-mediated cytotoxicity. This enables plasmid DNA to induce both humoral and cellular immune responses to the expressed proteins. The effect of the mucosal administration of DNA has not been extensively investigated although uptake of DNA from epithelial surfaces may not be as effective as direct injection of DNA into muscle.

### EXISTING STATE OF ART

When NMDAR antagonists are given to rodents in large doses, they can cause a form of brain damage called Olney's Lesions. However, there is insufficient research to show that large doses of NMDAR antagonists cause Olney's Lesions in humans and there are known to be fundamental differences between human and rodent brains. (Farber et al., 2003. Muscimol prevents NMDA antagonist neurotoxicity by activating GABAA receptors in several brain regions. Brain Res. 993: 90-100).

This single dose vaccine provided a strong anti-epileptic and neuroprotective activity in mice for both a kainate-induced seizure model and an endothelian-1 induced MCAO stroke model at 1 to 5 months following vaccination. Thus, a vaccination strategy targeting brain proteins is feasible and may have therapeutic potential for treatment of neurological disorders (During et al., 2000. An oral vaccine against NMDAR1 with efficacy in experimental stroke and epilepsy. Science 287:1453-1460).

In humans, there is an unusual predominance of IgA2 plasma cells in the lamina propria of the large intestine and in the female genital mucosal tissues (uterus, cervix, fallopian tubes, and vagina) (Crago et al., 1984. Distribution of IgA1-, IgA2-, and J chain-containing cells in human tissues. J. Immunol. 132:16-18 and Mestecky and Russell 1986. IgA subclasses. Monogr Allergy. 19:277-301).

Recently, attempts have been made to demonstrate the efficacy following oral immunization of NMDA-NR1 DNA to induce systemic and local immune responses and provide an anti-epilepsy and anti-stroke response in rats (During et al., 2000. An oral vaccine against NMDAR1 with efficacy in experimental stroke and epilepsy. Science 287:1453-1460).

Microparticles of less than 10 μm are readily taken up by intestinal M cells, macrophages and other professional antigen-presenting cells (APCs), leading to antigen presentation at regional inductive immune sites (Kim et al., 1999. Induction of mucosal and systemic immune response by oral immunization with H. pylori lysates encapsulated in poly (D, L-lactide-co-glycolide) microparticles. Vaccine. 17:607-616; Baras et al., 1999. Single-dose mucosal immunization with biodegradable microparticles containing a Schistosoma mansoni antigen. Infect Immun.; 67:2643-2648; Okada and Toguchi, 1995. Biodegradable microspheres in drug delivery. Crit. Rev Ther Drug Carrier Syst. 12:1-99).

Of these microparticles, PLGA has a long history of safe use in humans and has already been approved as a component of a number of drug-delivery systems (Klencke et al., 2002. Encapsulated plasmid DNA treatment for human papillomavirus 16-associated anal dysplasia: a Phase I study of ZYC101. Clin. Cancer Res. 8:1028-1037; Okada and Toguchi, 1995. see above).

It has been shown that PLGA-encapsulated plasmid DNA elicited systemic and mucosal antibodies to the encoded antigen, as well as cell mediated immune responses after oral delivery in non-primate and primate models (Kaneko et al., 2000. Oral DNA vaccination promotes mucosal and systemic immune responses to HIV envelope glycoprotein. Virology 267:8-16, Sharpe et al., 2003. Mucosal immunization with PLGA-microencapsulated DNA primes a SIV-specific CTL response revealed by boosting with cognate recombinant modified vaccinia virus Ankara. Virology. 313:13-21; Herrmann et al., 1999. Immune responses and protection obtained by oral immunization with rotavirus VP4 and VP7 DNA vaccines encapsulated in microparticles. Virology. 259:148-153; Singh et al., 2001. Mucosal immunization with HIV-1 gag DNA on cationic microparticles prolongs gene expression and enhances local and systemic immunity. Vaccine. 20:594-602).

NR1 genes were expressed in Baculovirus as per our and other previously published methods (Kawamoto et al., 1995. Expression and characterization of the zeta 1 subunit of the N-methyl-D-aspartate (NMDA) receptor channel in a baculovirus system. Brain Res Mol Brain Res. 30:137-148; Lenhard et al., 1996. A new set of versatile vectors for the heterologous expression of foreign genes using the baculovirus system. Gene (Amst.) 169: 187-190; Sydow et al., 1996. Overexpression of a functional NMDA receptor subunit (NMDAR1) in baculovirus-infected Trichoplusia ni insect cells. Brain Res Mol Brain Res. 41:228-40; Reddy et al., 1997. Application of recombinant bovine viral diarrhea virus proteins in the diagnosis of bovine viral diarrhea infection in cattle. Vet. Microbiol. 57:119-133 and Ivanovic et al., 1998. Expression and Initial Characterization of a Soluble Glycine Binding Domain of the N-Methyl-D-aspartate Receptor NR1 Subunit. J Biol Chem 273: 19933-19937).

The protein was purified using an anti-NR1 antibody-affinity column and the protein (53 kDa) was detected by radio-immunoprecipitation (RIP) using the methods described by Sydow et al., 1995 and Reddy et al., 1997.

A soluble recombinant r-NR1 protein was derived from insect cell expression, confirming the report by Ivanovic et al., 1998. Biochemical analysis of the produced protein revealed heterogeneity, because of N-linked glycosylation. Highly glycosylated protein was secreted into the insect cell medium. The glycosylation of NMDA receptor subunits in neurons is essential for correct targeting and subsequent secretion. Functional glycosylation of NR1 expressed in insect cells has been shown (Kawamoto et al., 1995; Ivanovic et al., 1998,).

Preparation of Microcapsules was Done According to the Methods Described by Yeo and Park, (Characterization of Reservoir-Type Microcapsules Made by the Solvent Exchange Method AAPS PharmSciTech 2004; 5:e52).

PLGA-NR1 DNA or r-protein nanoparticles with sizes less than 200 nm are designed to tightly bind DNA or protein with high efficiency. This small size is comparable to the acceptable size range for cationic particles that has been previously demonstrated to be effective (Tseng, 2001. Mechanics and multiple-particle tracking microheterogeneity of alpha-actinin-cross-linked actin filament networks. Biophys J. 81:1643-1656).

The release of NR1 showed a biphasic pattern of drug release characterized by a burst release followed by a slow release which is characteristic of matrix diffusion kinetics (Lemoine et al., 1998. Preparation and characterization of alginate microspheres containing model antigen. Int J Pharm, 176:9-19).

Before and after storage at 39.5° C./75% RH for 6 months, in vitro release data were analyzed for dissolution efficiency as per Costa and Sousa Lobo (2001. Modelling and comparison of dissolution profiles. Eur J Pharm Sci. 13:123-133).

Cationic PLGA-microparticles which carry protein or DNA have been shown by several workers to adsorb to mucous surfaces and to efficiently transfect cells in vitro (Denis-Mize, et al., 2000. Plasmid DNA adsorbed onto cationic microparticles mediates target gene expression and antigen presentation by dendritic cells. Gene Ther. 7:2105-2112; Singh et al., 2001. Mucosal immunization with HIV-1 gag DNA on cationic microparticles prolongs gene expression and enhances local and systemic immunity. Vaccine 20:594-602).

However, obtaining high transfection efficiencies in vivo is often limited by particle transport through extracellular barriers, including the mucosal barrier, which has been described as the foremost barrier to transfection in mucus-covered cells (Ferrari et al., 2001. Mucus altering agents as adjuncts for non-viral gene transfer to airway epithelium. Gene Ther. 8:1380-1386; Yonemitsu et al., 2000. Efficient gene transfer to airway epithelium using recombinant Sendai virus. Nat. Biotechnol. 18:970-973).

Mucus was formulated from 60 mg/ml PGM, 3.2 mg/ml DPPC, and 32 mg/ml BSA in sputum buffer (85 mM Na+, 75 mM Cl⁻, 20 mM Hepes, pH 7.4) (Sanders et al., 2000. Cystic fibrosis sputum: a barrier to the transport of nanospheres. Am. J. Respir. Crit. Care Med. 162:1905-1911).

Mucus was mixed on a stir plate for 48 h at 4° C. and stored at −20° C. as described. Reconstituted PGM had compositional and rheological properties physiologically relevant to gastrointestinal (GI) mucus (Khanvilkar et al., 2001. Drug transfer through mucus. K Adv Drug Deliv Rev. 48:173-193).

CMI responses were measured by the procedures described earlier (Furesz et al., 1997. Antibody- and cell-mediated immune responses of Actinobacillus pleuropneumoniae-infected and bacterin-vaccinated pigs. Infect Immun. 65:358-365 and Reddy et al., 1999. Semiliki forest virus vector carrying the bovine viral diarrhea virus NS3 (p80) cDNA induced immune responses in mice and expressed BVDV protein in mammalian cells. Com Imm Micro Infec Dis. 22: 31-246).

There were no detectable cell mediated immune responses after vaccination with NMDA-NR1 r-protein or DNA as measured by the procedures described earlier (Reddy et al., 1999. Semiliki forest virus vector carrying the bovine viral diarrhea virus NS3 (p80) cDNA induced immune responses in mice and expressed BVDV protein in mammalian cells. Com Imm Micro Infec Dis. 22: 31-246).

Studies have shown that the inhibition of lipid peroxidation attenuates neuronal damage in animal models of cerebral ischemia and reduces brain infarct volume in a MCAO model of stroke, when NR1 was administered to animals which were not vaccinated with NR1 (During et al., 2000. An oral vaccine against NMDAR1 with efficacy in experimental stroke and epilepsy. Science 287:1453-1460).

In addition, blockade of Na+ channels has been proposed as a possible neuroprotective mechanism by reducing energy expenditure in compromised tissue (Adkins et. al., 2004. behavioral and neuro-plastic effects of focal endothelin-1 induced sensori motor cortex lesions. Neuroscience 128: 473-486; Urenjak and Obrenovitch 1996. Pharmacological modulation of voltage-gated Na+ channels: a rational and effective strategy against ischaemic brain damage. Pharmacol Rev. 48:21-67; Callaway et al., 1999. Delayed Treatment With AM-36, a Novel Neuroprotective Agent, Reduces Neuronal Damage After Endothelin-1-Induced Middle Cerebral Artery Occlusion in Conscious Rats. Stroke. 30:2704-2712)

Neurological abnormalities were evaluated with using a neurological deficit score based on detection of abnormal posture and hemiplegia, as described by De Ryck et al. (1989. Photochemical stroke model: flunarizine prevents sensorimotor deficits after neocortical infarcts in rats. Stroke 20:1383-1390).

Sensory hemi-neglect in pigs was evaluated by a test that measures sensitivity to simultaneous forelimb stimulation (Schallert and Whishaw, 1984. Bilateral cutaneous stimulation of the somatosensory system in hemi-decorticated pigs. Behav Neurosci. 98:518-540).

The sensory hemi-neglect test is based on observations of behavior in humans with unilateral brain damage (Daffner et al., 1990. Dissociated neglect behaviour following sequential strokes in the right hemisphere. Ann Neurol. 28:97-101; Ferro et al., 1987. Subcortical neglect: quantitation, anatomy, and recovery. Neurology. 37:1487-1492).

The volume of brain damage was determined as described previously (Park et al., 1989. Effect of the NMDA antagonist MK-801 on local cerebral blood flow in focal cerebral ischaemia in the rat. J Cereb Blood Flow Metab. 9:617-622; Sharkey and Butcher, 1994. Immunophilins mediate the neuroprotective effects of FK506 in focal cerebral ischaemia. Nature. 371:336-9).

Briefly, the area of brain damage of eight selected brains was assessed using light microscopy by an observer who was unaware of the treatment groups. The volume of brain damage was calculated by integration of the cross-sectional area of damage at each stereotaxic level and the distances between the various levels (Park et al., 1989; Sharkey and Butcher, 1994, see above).

All procedures used in this study were performed in accordance with the Prevention of Cruelty to Animals Act.

A 23-gauge stainless steel guide cannula was stereotaxically implanted into the piriform cortex 2 mm dorsal to the right MCA, according to the method of Sharkey et al., (1993. Perivascular microapplication of endothelin-1: a new model of focal cerebral ischemia in the rat. J Cereb Blood Flow Metab. 13:865-871). The stereotaxic coordinates were modified (0.2 mm anterior, −5.2 mm lateral and −6.1 mm ventral, according to a stereotaxic atlas (De Ryck et al., 1989. Photochemical stroke model: flunarizine prevents sensorimotor deficits after neocortical infarcts in rats. Stroke. 20:1383-1390).

Total infarct volume was calculated by integrating the cross-sectional area of damage at each stereotaxic level and the distances between the levels according to the method of Osborne et al., (1987. Quantitative assessment of early brain damage in a rat model of focal cerebral ischaemia. J Neurol Neurosurg Psychiatry. 50:402-410).

Histopathology performed on the brains 80 hours after injection of ET-1 showed a pattern of damage similar to that found in other animal species in previous reports (During et al., 2000. An oral vaccine against NMDAR1 with efficacy in experimental stroke and epilepsy. Science 287:1453-1460; Sharkey et al., 1993. Perivascular microapplication of endothelin-1: a new model of focal cerebral ischaemia in the rat. J Cereb Blood Flow Metab. 13:865-871) which included consistent lesions in the parietal and insular cortex, variable degrees of damage in the frontal cortex, and infarction most often in the dorso-lateral striatum but sometimes extending throughout the corpus striatum.

The temperature drop during the period of stroke induction might be due to anesthesia as reported in some pig studies (Hensel et al., 1995. Oral immunization of pigs with viable or inactivated Actinobacillus pleuropneumoniae serotype 9 induces pulmonary and systemic antibodies and protects against homologous aerosol challenge. Infect Immun. 63:3048-3053).

Research has indicated a relationship between stages of neuropathological degeneration and clinical diagnosis suggesting that the entorhinal cortex shows early signs of memory loss linked to the hippocampus (Braak, H. and Braak, E. 1991. Neuropathological staging of Alzheimer-related changes. Acta Neuropathologica 82: 239-259; Jack et al., 1992. MR-based hippocampal volumetry in the diagnosis of Alzheimer's disease. Neurology 42:183-188). It was reported that the volume of the hippocampi in affected subjects was significantly reduced in comparison to healthy controls.

Further, pathological studies of smaller hippocampi and entorhinal cortices among individuals with neurological disorders have supported these observations (Juottonen, et al., 1999. Comparative MR analysis of the entorhinal cortex and hippocampus in diagnosing Alzheimer's disease. AJNR Am J. Neuroradiol. 20:139-144).

Allocentric place memory may serve to specify the context of events stored in human episodic memory. The same neurobiological substrates relevant to associations of episodic memory in animals could be associated with human episodic memory. An example is the memory of single specific events of places where animals find food with preferred flavor as reported by Tulving (2002. Episodic memory: from mind to brain. Annu Rev Psychol 53:1-25).

Blockade of remembered events has been reported by several workers (Gaffan 1991. Spatial organization of episodic memory. Hippocampus 3:262-264; Burgess et al., 2002. The human hippocampus and spatial and episodic memory. Neuron 35:625-641; Buzsaki et al., 1986. Laminar distribution of hippocampal rhythmic slow activity (RSA) in the behaving rat: current-source density analysis, effects of urethane and atropine. Brain Res 365:125-137).

Allocentric place memory requires fast excitatory transmission through hippocampal synapses, essentially mediated by AMPA receptors (Davies S N, Collingridge G L (1989) Role of excitatory amino acid receptors in synaptic transmission in area CA1 of rat hippocampusm Proc R Soc Lond B Biol Sci 236:373-384; Lambert J D C, Jones R S G (1990) A re-evaluation of excitatory amino acid-mediated synaptic transmission in rat dentate gyrus. J Neurophysiol 64:119-132), to activate the stored place. Alternatively, it has been suggested that the neural representation of trial-specific places in familiar environments over minutes to hours may not require hippocampal NMDA receptors (Shapiro M L, O'Connor C (1992) N-methyl-D-aspartate receptor antagonist MK-801 and spatial memory representation: working memory is impaired in an unfamiliar environment but not in a familiar environment. Behav Neurosci 106:604-612; Caramanos Z, Shapiro M L (1994) Spatial memory and N-methyl-D-aspartate receptor antagonists APV and MK-801: memory impairments depend on familiarity with the environment, drug dose, and training duration. Behav Neurosci 108:30-43; Kesner R P, Rolls E T (2001) Role of long-term synaptic modification in short-term memory. Hippocampus 11:240-250. Studies have demonstrated that, analogous to event-place associations in episodic memory, rats could associate, within one trial, a specific food flavor with an allocentrically defined place in an open arena (Bast et al., 2005. Distinct Contributions of Hippocampal NMDA and AMPA Receptors to Encoding and Retrieval of One-Trial Place Memory J. Neurosci. 25:5845-5856). Encoding, but not retrieval, of such flavor-place associations required hippocampal NMDA receptors; retrieval depended on hippocampal AMPA receptors.

The values for the correct choice expected based on chance were 20% of the first choices or 20% of the total dig time at each food-well and an average number of two errors per trial as reported (Clayton and Dickinson 1998. Episodic-like memory during cache recovery by scrub jays. Nature 395:272-274; Clayton and Krebs 1994. One-trial associative memory: comparison of food storing and nonstoring species of birds. Anim Learn Behav. 22:366-372. Clayton et al., 2003. Can animals recall the past and plan for the future? Nat Rev Neurosci 4:685-691).

Recently developed episodic-like memory tasks in animals, requiring integrated one-trial memory of events, their places, and their temporal context, depend on the hippocampus (Clayton N S, Bussey T J, Dickinson A (2003) Can animals recall the past and plan for the future? Nat Rev Neurosci 4:685-691; Day M, Langston R, Morris R G M (2003) Glutamate-receptor-mediated encoding and retrieval of paired-associate learning. Nature 424:205-209; Eacott M J, Norman G (2004) Integrated memory for object, place, and context in rats: a possible model of episodic-like memory? J Neurosci 24:1948-1953. Eichenbaum H (2004) Hippocampus: cognitive processes and neural representations that underlie declarative memory. Neuron 44:109-120) similar to human episodic memory (Tulving 2002. Episodic memory: from mind to brain. Annu Rev Psychol 53:1-25.).

The contributions of hippocampal NMDA receptors to one-trial place memory is probably important for retention of episodic-like memories and may partly explain the requirement of these receptors for one-trial flavor-place memory (Day et al., 2003. Glutamate-receptor-mediated encoding and retrieval of paired-associate learning. Nature 424:205-209).

However, hippocampal NMDA receptors are critical for rapid encoding of relational memory without a place component (Roberts M, Shapiro M L (2002) NMDA receptor antagonists impair memory for nonspatial, socially transmitted food preference. Behav Neurosci 116:1059-1069) and may generally contribute to binding distinct aspects of episodic-like memory, such as flavor and place information, into relational representations (Eichenbaum 2004. Hippocampus: cognitive processes and neural representations that underlie declarative memory. Neuron 44:109-120).

The hippocampal dorsoventral differentiation could help in dissecting the different hippocampal contributions to episodic-like memory. For example, the dorsal hippocampus, receiving most of the hippocampal visuospatial afferents, may be more important than the ventral hippocampus for encoding one-trial place memory (Bannerman et al., 1995. Distinct components of spatial learning revealed by prior training and NMDA receptor blockade. Nature 378:182-186.).

In contrast, with substantial olfactory and gustatory input reaching the ventral hippocampus, dorsoventral interactions may be required to bind place and flavor information (Petrovich et al., 2001. Combinatorial amygdalar inputs to hippocampal domains and hypothalamic behavior systems. Brain Res Rev 38:247-289).

Mean Arterial Blood Pressure (MABP) was unaffected by NMDA-NR1 pre or post-treatment. Although body and brain temperature influence the severity of brain damage after focal cerebral ischemia (Morikawa et al., 1992. The significance of brain temperature in focal cerebral ischemia: histopathological consequences of middle cerebral artery occlusion in the rat. J Cereb Blood Flow Metab. 12:380-389; Xue et al., 1992. Immediate or delayed mild hypothermia prevents focal cerebral infarction. Brain Res. 587:66-72), these variables were unaltered following ET-1 induced MCAO.

Some NMDA antagonists, for example the pharmaceutical drugs MK801 and nitroglycerol, have a very narrow window of opportunity in focal ischemia models and are ineffective if not administered within 30 minutes of the onset of the stroke (Liu, 1993. FK506 and cyclosporin: molecular probes for studying intracellular signal transduction. Trends Pharmacol Sci 14:182-188; Liu et al., 1991. Calcineurin is a common target of cyclophilin-cyclosporin A and FKBP-FK506 complexes. Cell 66:807-815). In contrast, Na⁺ channel antagonists and antioxidants appear to have a longer window of opportunity. For example, α-phenyl-tert-butyl nitrone (PBN) reduced brain infarct volume in a rat MCAO model of stroke when administered up to 3 hours after ischemia (Chen, 1994. Asymmetrical blockade of the Ca2+ release channel (ryanodine receptor) by 12-kDa FK506 binding protein. Proc Natl Acad Sci USA 91:11953-11957; Chiu et al., 1994. RAPT1, a mammalian homolog of yeast Tor, interacts with the FKBP12/rapamycin complex. Proc Natl Acad Sci USA 91:12574-12578). ET-1 (120 pmol in 3 μl) resulted in blood flows of <25 ml/100 g per minute (<20% of normal), which were still evident in the striatum and sensory cortex at 3 hours after injection (Estévez, et al., 1995. Peroxynitrite-induced cytotoxicity in PC12 cells: Evidence for an apoptotic mechanism differentially modulated by trophic factors. J. Neurochem, 65: 1543-1550).

Hence, it is possible that the greater effectiveness of NMDA-NR1 antibodies may reflect the greater production of reactive oxygen species associated with re-perfusion at this time. Alternatively, maintenance of ionic homeostasis through the ability of PBN to block Na⁺ channels, may dictate a critical period. Previous studies showed the effectiveness of delayed administration of pharmaceuticals such as Na⁺ channel antagonists (Brown et al., 1994. A mammalian protein targeted by GI-arresting rapamycin-receptor complex. Nature. 369:756-758; Dawson et al., 1993. Immunosupressant, FK-506, Enhances Phosphorylation of Nitric Oxide Synthase Against Glutamate Neurotoxicity. Proc. Natl. Acad. Sci., USA, 90:9808-9812).

Embolic stroke in humans leads to lesions and consequent behavioral deficits, including language and motor dysfunction (Liu et al., 1994. Calcineurin inhibition of dynamin I GTPase activity coupled to nerve terminal depolarization. Science 265:970-973).

Sensory hemi-neglect is a phenomenon that has been reported during the course of recovery from stroke in patients with damage to the striatum and in patients with right parietal lobe infarction Li Y, Sharov V G, Jiang N, Yao F, Zaloga C, Sabbah H N, Chopp M. 1995: “Ultrastructural and light microscopic evidence of apoptosis after middle cerebral artery occlusion in rat.” Am J Pathol; 146:1045-1051.

One study with FK506 produced a 78% anticonvulsant effect, which compares favorably with previous pharmacological studies in which similar levels of anti-stroke activity were typically associated with significant motor impairment (Gundersen et. al., 1988. The new stereological tools: Disector, Fractionator, nucleator, and point sampled intercepts and their use in pathological research and diagnosis. APMIS 96:857-881; Butcher et al., 1997. Neuroprotective actions of FK506 in experimental stroke: in vivo evidence against an antiexcitotoxic mechanism. J. Neurosci. 17:6939-6846).

However, even with this antagonist at doses that depress motor activity, tissue rescue is limited to 50% in the cortex with no infarct reduction in the striatum (Butcher et al., 1997. see above; Steinberg et al., 1995. Neuroprotection by N-methyl-D-aspartate antagonists in focal cerebral ischemia is dependent on continued maintenance dosing. Neuroscience 64, 99-107), A major limitation of the successful translation of promising NMDA-receptor antagonists to the clinic has been the significant profile of adverse effects affecting the central nervous system (Schehr, 1996. New treatments for acute stroke. Nature Biotechnol. 14:1549-1554).

A potential advantage of a vaccine or antibody as an approach to NMDA antagonism is that blockade of the receptor is minimal under resting physiological conditions under which high serum titers of antibodies do not pass the blood brain barrier efficiently. However, after a neuronal insult, the blood brain barrier shows increased permeability to serum antibodies, and transport and subsequent binding to the target protein can occur (Aihara, et al., 1994. Immunocytochemical localization of immunoglobulins in the rat brain: relationship to the blood-brain barrier. J. Comp. Neurol. 342:481-496). Glutamate itself has been reported to alter the permeability of the blood brain barrier (W. G. Mayhan and S. P. Didion, 1996. Glutamate-induced disruption of the blood-brain barrier in rats. Role of nitric oxide. Stroke 27:965-969).

Protective efficacy of NMDA-NR1 for epilepsy was already shown by During, et al., 2000 (An oral vaccine against NMDAR1 with efficacy in experimental stroke and epilepsy. Science 287:1453-1460) in a kainate-induced epilepsy model in rats.

### BRIEF SUMMARY OF THE INVENTION

The purpose of this work was to assess the ability of plasmid DNA encoding NMDA-NR1 protein and NR1 DNA, encapsulated in poly (DL-lactide-co-glycolic acid) (PLGA) microparticles, to induce local and systemic NR1-specific immunity following a single dose of oral immunization. Oral administration of PLGA-NMDA-NR1 protein or DNA microparticles induced a long-lasting and stable antigen-specific serum antibody response in pigs, including both IgG and IgA. Immunized pigs exhibited antigen-specific humoral immune responses but not cell mediated immune responses. Immunization with both types of NR1-PLGA microparticles produced a humoral immune response detected by serum antibody reactivity to NR1 r-protein in isotype-specific ELISA. The results are encouraging with regard to obtaining good compliance and vaccination coverage with the candidate r-NR1 protein. In unvaccinated animals, our results with post-stroke treatment in animals indicate that the r-NR1 protein also shows promise as therapy after a stroke occurs.

The most attractive route for mucosal immunization is the oral route because it is painless. It results in high patient compliance, coupled with ease of administration and applicability to mass vaccination. Recently, attempts have been made to demonstrate efficacy following oral immunization of NMDA-NR1 DNA to induce systemic and local immune responses and provide an anti-epilepsy and anti-stroke response in rats (During et al., 2000. An oral vaccine against NMDAR1 with efficacy in experimental stroke and epilepsy. Science 287:1453-1460). Microparticles of less than 10 μm are readily taken up by intestinal M cells, macrophages and other professional antigen-presenting cells (APCs), leading to antigen presentation at regional inductive immune sites (Kim et al., 1999. Induction of mucosal and systemic immune response by oral immunization with H. pylori lysates encapsulated in poly (D, L-lactide-co-glycolide) microparticles. Vaccine. 17:607-616; Baras et al., 1999. Single-dose mucosal immunization with biodegradable microparticles containing a Schistosoma mansoni antigen. Infect Immun.; 67:2643-2648; Okada & Toguchi, 1995. Biodegradable microspheres in drug delivery. Crit. Rev. Ther. Drug Carrier Syst. 12:1-99). Of these microparticles, PLGA has a long history of safe use in humans and has already been approved as a component of a number of drug-delivery systems (Klencke et al., 2002. Encapsulated plasmid DNA treatment for human papillomavirus 16-associated anal dysplasia: a Phase I study of ZYC101. Clin. Cancer Res. 8:1028-1037; Okada and Toguchi, 1995. see above). It has been shown that PLGA-encapsulated plasmid DNA elicited systemic and mucosal antibodies to the encoded antigen, as well as cell mediated immune responses after oral delivery in non-primate and primate models (Kaneko et al., 2000, Oral DNA vaccination promotes mucosal and systemic immune responses to HIV envelope glycoprotein. Virology 267:8-16, Sharpe et al., 2003. Mucosal immunization with PLGA-microencapsulated DNA primes a SIV-specific CTL response revealed by boosting with cognate recombinant modified vaccinia virus Ankara. Virology. 313:13-21; Herrmann et al., 1999. Immune responses and protection obtained by oral immunization with rotavirus VP4 and VP7 DNA vaccines encapsulated in microparticles. Virology. 259:148-153; Singh et al., 2001. Mucosal immunization with HIV-1 gag DNA on cationic microparticles prolongs gene expression and enhances local and systemic immunity. Vaccine. 20:594-602).

### BRIEF DESCRIPTION OF THE SEVERAL VIEWS OF THE DRAWINGS

FIG. 1. The chart showing the optimization extent of codon quality to suit expression requirements of both Escherichia coli (CAI: 0.84) and Spodoptera frugiperda (Sf9) (CAI: 0.82).

FIGS. 2 & 3. showing the GC content before and after optimization.

FIG. 4. showing the changes made to the human NMDA-NR1 sequence during optimization. Sequence from GenBank (L05666.1) is the top line and alterations are indicated in the lower line. Start and stop codons are underlined. Amino acid sequences are identical.

FIG. 5. showing the NMDA-NR1 subunit receptor cloned into the pAAV-MCS vector.

FIG. 6. showing the scanning electron microscopy photograph of PLGA microsphere which contains 100 μg r-NR1 DNA/mg microparticles.

FIG. 7. showing the Scanning electron microscopy photograph of PLGA microparticles with cores containing the NMDA-NR1 r-protein formulation of 200 μg/mg microparticles.

FIG. 8. showing the in vitro release studies. Data were fitted into various release equations to explain the kinetics of drug release of NR1 r-protein or DNA from these microspheres. The examination of the R2 coefficient indicated that vaccine release from the core of the coated PLGA microspheres followed the diffusion control mechanism. To explore the kinetic behavior, in vitro release results were further fitted into the following Korsmeyer and Peppas equation: M t M ∞=K t n, where Mt/M∞ is the fraction of drug released after time t, K is a kinetic constant, and n is a release exponent that characterizes the drug transport.

FIG. 9. showing the percentage of NR1 protein released from the microsphere formulation before and after storage at 39.5° C./75% relative humidity for 6 months.

FIG. 10. is the chart showing the Differential Scanning Calorimetry (DSC) thermogram of % Recovery of NR1 protein, DNA or PLGA stored at 39.5° C./75% relative humidity for 10-210 days (24 months).

FIG. 11. the 3D chart showing NMDA-NR1 r-protein time of treatment verses damage: Neuroprotective efficacy of NMDA-NR1 against endothelin-1-induced MCAO reduction of cortical volume of brain damage (mm³) of swine.

FIG. 12. is the chart showing the EEG of minor seizures with an abrupt ending of electroencephalographic seizure activity within 10 seconds after induction with ET-1 (EEG: Seizure free interval showing no tendency of stroke activity in NMDA r-protein or DNA immunized pigs).

FIG. 13. is the chart showing EEG of a pig with a minor neurological deficit (mild right-side hemiparesis, NIHSS=3) upon treated with NMDA-NR1, 30 minutes after MCAO. (EEG: Seizure free interval with interictal spikes in NMDA NR-1 treated pigs).

FIG. 14. is the chart showing EEG of a pig with a right cerebral infarct (NIHSS=13) showing polymorphic delta activity and development of subtle symptomatic focal seizures. (EEG: Stroke Seizures in Placebo pigs).

FIG. 15. is the chart showing the rectal temperatures taken from NR1 vaccinated pigs and control pigs.

FIG. 16. is the chart showing the brain temperatures (intra-cerebral temperatures (striatal) temperature (° C.) taken from immunized pigs.

### DETAILED DESCRIPTION OF THE INVENTION

### Detailed Description of the Preferred Embodiment I

NMDAR is a protein on the surface of neurons (nerve cells). When the major excitatory neurotransmitter, glutamate, binds to this protein, the central pore of the NMDAR channel opens to allow the cations sodium, potassium, and calcium to cross the cell membrane. The movement of these cations through the pore results in neuronal excitation. The NMDAR is one of several cell-surface receptor proteins which are activated by glutamate. It is believed that over activation of the NMDA receptor could be responsible for the neuronal cell death which is observed following some forms of stroke; it may even be involved in the cell-death associated with neurodegenerative diseases.

The NMDAR is an important glutamate receptor that acts as a non-selective cation channel which is highly permeable to both calcium (Ca2+) and sodium (Na+). The activation of NMDA receptors results in prolonged increases of intracellular Ca2+ concentration and thereby triggers downstream signaling pathways which are involved in the regulation of many physiological and pathophysiological processes. Previous studies have focused on how Ca2+ or Na+ affects NMDAR activity in isolation. Specifically, the increase in intracellular Ca2+ concentration may down regulate NMDA channels and may act as a negative feedback mechanism controlling NMDAR activity, whereas an increase in intracellular Na+ concentration may up regulate NMDAR channel activity. A critical question that has yet to be answered is how an individual NMDAR may be regulated when both of these ionic species flow into neurons during the same time period via neighboring, activated NMDARs.

The gating of a NMDAR channel has been reported to be regulated by the activation of remote NMDARs via an interaction between Na+ and Ca2+. During the activation of NMDARs, the influx of Na+ has been reported to potentiate Ca2+ influx on the one hand and to overcome Ca2+-induced inhibition of NMDAR channel gating on the other hand. Furthermore, the intracellular concentration of Na+ is able to mask the effects of Ca2+ on NMDAR channel gating in cultured hippocampal neurons. There appears to be cross talk between neighboring NMDARs mediated by a functional Na+-Ca2+ interaction via a novel mechanism which regulates NMDAR activity. This study demonstrates that the novel neuroprotective NMDA-NR1 r-protein or DNA vaccine or NMDA-NR1 r-protein treatment greatly attenuates both cortical and striatal damage and also improves functional outcome after MCAO in pigs. A significant linear trend was seen for greater histological improvement with decreased delay in time of administration of NR1 r-protein.

A similar but not statistically significant trend was observed for striatal damage, although there was marked reduction in striatal damage found in pigs treated with NMDA-NR1 r-protein up to 80 minutes after stroke. The striatum is generally considered to be the core of the ischemic lesion and previously has proved relatively refractory to neuroprotection. Some NMDA antagonists, for example the pharmaceutical drugs MK801 and nitroglycerol, have a very narrow window of opportunity in focal ischemia models and are ineffective if not administered within 30 minutes of the onset of the stroke (Liu, 1993. FK506 and cyclosporin: molecular probes for studying intracellular signal transduction. Trends Pharmacol Sci 14:182-188; Liu et al., 1991. Calcineurin is a common target of cyclophilin-cyclosporin A and FKBP-FK506 complexes. Cell 66:807-815.).

In contrast, Na⁺ channel antagonists and antioxidants appear to have a longer window of opportunity. For example, α-phenyl-tert-butyl nitrone (PBN) reduced brain infarct volume in a rat MCAO model of stroke when administered up to 3 hours after ischemia (Chen, 1994. Asymmetrical blockade of the Ca2+ release channel (ryanodine receptor) by 12-kDa FK506 binding protein. Proc Natl Acad Sci USA 91:11953-11957; Chiu et al., 1994. RAPT1, a mammalian homolog of yeast Tor, interacts with the FKBP12/rapamycin complex. Proc Natl Acad Sci USA 91:12574-12578). ET-1 (120 pmol in 3 μL) resulted in blood flows of <25 ml/100 g per minute (<20% of normal), which were still evident in the striatum and sensory cortex at 3 hours after injection (Estévez et al., 1995. Peroxynitrite-induced cytotoxicity in PC12 cells: Evidence for an apoptotic mechanism differentially modulated by trophic factors. J. Neurochem, 65:1543-1550). Hence, it is possible that the greater effectiveness of NMDA-NR1 antibodies may reflect the greater production of reactive oxygen species associated with reperfusion at this time.

Alternatively, maintenance of ionic homeostasis through the ability of PBN to block Na⁺ channels, may dictate a critical period. Previous studies showing the effectiveness of delayed administration of pharmaceuticals such as Na⁺ channel antagonists (Brown et al., 1994. A mammalian protein targeted by GI-arresting rapamycin-receptor complex. Nature. 369:756-758; Dawson et al., 1993. Immunosupressant, FK-506, Enhances Phosphorylation of Nitric Oxide Synthase Against Glutamate Neurotoxicity. Proc. Natl. Acad. Sci., USA. 90:9808-9812). Whereas, our findings indicate that delaying treatment increases the brain damage.

A particularly preferred embodiment is a vaccine composition comprising nucleic acid encoding NMDA NR1 or recombinant protein expressed by insect cells.

Methods of making and using the compositions described herein are also embodiments of the invention to methods of making the embodied nucleic acids and protein expressed by insect cells using genetic vector cloned with NMDA NR1 genes. The embodiments include methods of making vaccine compositions that can be used to treat or prevent Stroke. Some methods are practiced, for example, by encapsulating in PLGA particles, as described so as to formulate a single composition (e.g., a vaccine composition). Preferred methods involve the encapsulation of NMDA NR1 DNA or recombinant antigen disclosed herein.

Preferred methods of using the compositions described herein involve providing an animal in need of a potent response to Stroke with a sufficient amount of the nucleic acid or protein embodiments described herein. By one approach, for example, animals vaccinated with NMDA NR1 r-protein or DNA potent in protection against artificial induction stroke or NMDA NR1 r-protein treatment and that is sufficient to enhance or facilitate an protective response to said antigen. In some embodiments, the composition described above also contains an amount of PLGA that retained a long term vaccine effect against stroke.

The possibility that NMDA-NR1 exerted its effects by lowering the body temperature of the animals was discounted because rectal temperatures did not differ between treatment groups after ischemia, and NMDA-NR1 r-protein or DNA, either in treatment or administered as a vaccine, had no effect on temperature. It is therefore important to demonstrate improvement in functional outcome as well as in histological improvement when neuroprotective agents are tested. For example, embolic stroke in humans leads to lesions and consequent behavioral deficits, including language and motor dysfunction (Liu et al., 1994. Calcineurin inhibition of dynamin I GTPase activity coupled to nerve terminal depolarization. Science 265:970-973). This result parallels the trend for greater histological improvement with decreased delay in time of administration of NMDA-NR1 r-protein and indicates protection against motor impairments induced by MCAO in pigs.

Sensory hemi-neglect is a phenomenon that has been reported during the course of recovery from stroke in patients with damage to the striatum and in patients with right parietal lobe infarction (Li et al., 1995: Ultrastructural and light microscopic evidence of apoptosis after middle cerebral artery occlusion in rat. Am J Pathol; 146:1045-1051). If two stimuli are presented simultaneously, one on each side of the body, the contra-lateral stimulus appears to be masked (extinguished) and remains undetected until the ipsilateral stimulus is removed. Patients report that they can feel both stimuli, although the contra lateral stimulus feels subjectively weaker than the ipsilateral stimulus. Sensory hemi-neglect was evaluated in the present study using a test developed for pigs. Control pigs showed a consistent sensory hemi-neglect on the side contra lateral to the MCAO, as indicated by an increased latency to both touch and to remove a stimulus placed on the contra lateral forelimb. Latency to touch and removal of a stimulus simultaneously placed on the ipsilateral side was unaffected by ischemia.

Treatment with NMDA-NR1 at 30 minutes after stroke effectively removed the hemi-neglect observed in control pigs and returned touch and removal latency to pre-surgery levels. The effect of NMDA-NR1-r-protein, when administered within 30 minutes after stroke, was reflected in the protection found in the striatum. The greatest improvement in this test occurs in those groups that show the greatest reductions in infarction volume. The functional test results correlate well with histological outcome. Improvement in neurological deficit scores paralleled the findings in the hemi-neglect test.

NMDA-receptor antagonists have been reported to influence several experimental animal models of human neurological disease. The systemic administration of ET-1 is a well established stroke model. Moreover, previous studies have shown that NMDA-receptor antagonism can partially inhibit the severity of stroke in this model in which ET-1 induces excessive glutamate release by activation of both pre- and post-synaptic receptors, and in which NMDA mediated neuronal excitation and injury occur. One study produced a 78% anticonvulsant effect, which compares favorably with previous pharmacological studies in which similar levels of anti-stroke activity were typically associated with significant motor impairment (Gundersen et. al., 1988. The new stereological tools: Disector, Fractionator, nucleator, and point sampled intercepts and their use in pathological research and diagnosis. APMIS 96:857-881; Butcher, et. al., 1997. Neuroprotective actions of FK506 in experimental stroke: in vivo evidence against an antiexcitotoxic mechanism. J. Neurosci. 17: 6939-6846). However, even with this antagonist at doses that depress motor activity, tissue rescue is limited to 50% in the cortex with no infarct reduction in the striatum (Butcher, et. al., 1997. see above; Steinberg et al., 1995. Neuroprotection by N-methyl-D-aspartate antagonists in focal cerebral ischemia is dependent on continued maintenance dosing. Neuroscience 64:99-107), in contrast to our results with the genetically engineered NMDA-NR1 protein where less than 10% damage occurred for a short period of time. Moreover, there is a narrow time window of only a 30-min in which NMDA-NR1 protein seems to be a promising new anti-stroke drug which can be given for rescue, providing effective protection with no requirement of continued maintenance dosing.

The NMDA-NR1-vaccinated pigs generated polyclonal autoantibodies. Several animals did not appear to have an immunodominant epitope, a result which is consistent with antibodies that are dependent on the conformational state of the protein. Further analysis of epitopes and protein fragments and correlation with neuroprotection will be necessary to define which sites of the NMDA-NR1 protein are the best targets for neuronal protection and vaccine development.

A major limitation of the successful translation of promising NMDA-receptor antagonists to the clinic has been the significant profile of adverse effects affecting the central nervous system (Schehr, 1996. New treatments for acute stroke. Nature Biotechnol. 14:1549-1554).

A potential advantage of a vaccine or antibody as an approach to NMDA antagonism is that blockade of the receptor is minimal under resting physiological conditions under which high serum titers of antibodies do not pass the blood brain barrier efficiently. However, after a neuronal insult, the blood brain barrier shows increased permeability to serum antibodies, and transport and subsequent binding to the target protein can occur (Aihara, et al., 1994. Immunocytochemical localization of immunoglobulins in the rat brain: relationship to the blood-brain barrier. J. Comp. Neurol. 342:481-496). Glutamate itself has been reported to alter the permeability of the blood brain barrier (Mayhan and Didion, 1996. Glutamate-induced disruption of the blood-brain barrier in rats. Role of nitric oxide. Stroke 27:965-969). This suggests that our vaccine strategy might induce an autoprotective loop, whereby a cerebral insult increases the concentration of extracellular glutamate in the brain which leads to a local increase in the blood brain barrier permeability, resulting in facilitated passage of the autoantibody and antagonism of glutamate receptors.

We showed neuroprotection in the pigs immunized against NMDA-NR1 but not in the control groups. Moreover, the induction of autoantibodies that bound functional domains of the NMDAR and that yielded a staining pattern in hippocampal slices confined to cells known to express native NR1 receptors suggests that the immunization effect was specific to the NMDA-NR1. Furthermore, the passage into the CNS of antibodies binding to other neuronal surface proteins might also protect against neurotoxic insults caused not only by stroke but also neuronal injury associated with neurological disorders such as epilepsy, Alzheimer, Parkinson, and Huntington diseases in humans. In contrast to the motor impairment associated with systemic administration of most pharmacological NMDAR blockers, the pigs vaccinated with AAV-NMDA-NR1 showed no impairment in motor function. Our results therefore suggest the possibility of vaccination for individuals at risk of stroke and other cerebral insults found in epilepsy, Alzheimer, Parkinson, and Huntington diseases without impairment of neurological function. Protective efficacy of NMDA-NR1 for epilepsy was already shown by During, et al., 2000 (An oral vaccine against NMDAR1 with efficacy in experimental stroke and epilepsy. Science 287:1453-1460) in a kainate-induced epilepsy model in rats.

Oral immunization with recombinant NMDA-NR1 produced a humoral response which persisted over many months and may possibly persist for years. One possible limitation is that the chronic elevation of these autoantibodies may eventually have undesirable effects on brain function. We are looking at the long term effects of the immunization on more complex behaviors mediated by NMDA-NR1. These experiments and additional safety studies must be investigated before any translation of this technology to the clinic. The clinical utility of current anti-stroke drugs has been limited by a narrow therapeutic time window for rescue and clinical benefit. A stroke vaccine that generates autoantibodies whose access to the brain and neuroprotective activity is spatially and temporally regulated may hold promise as a prophylactic measure to protect the brain. Moreover, the ability of systemic immunization to generate autoantibodies which bind to, and thereby alter the function of native brain proteins, opens new possibilities for modulating the nervous system and treating psychiatric disorders. It may not be limited to stroke treatment and prevention, but could be a promising treatment or cure for other neurological disorders such as epilepsy, Alzheimer, Parkinson, and Huntington diseases.

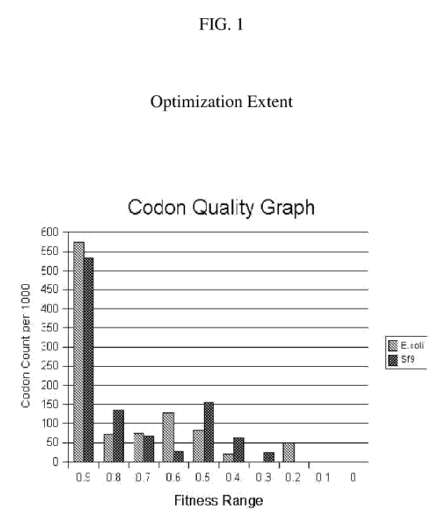

NMDA-NR1 Synthesis and Optimization: Sequence L05666 was optimized for codon utilization, secondary structures, repeats and other motifs (FIG. 1). A total of 665 codons (25%) were changed during the entire processing.

Optimization Input

### Sequence Description:

| | |
|---|---|
| Sequence Name | L05666 |
| Sequence Type | DNA |
| Length | 3245 bp |
| Accession | L05666 |
| Locus | HUMNMDAREC |
| Organism | Homo sapiens (human) |
| Description | Homo sapiens NMDA receptor subunit (NR1) |
| | mRNA, complete cds. |

Sequence Parameters

| | |
|---|---|
| Frame of Interest | +3 |
| ORF of Interest | 18 to 2675 |

Codon Usage Parameters:

| | |
|---|---|
| Intended Host Codon Frequency Table | Ecoli_Sf9 |
| Intended Host Genetic Codon Table | Standard Code |
| Codon Cutoff Frequency | 0.10 |
| Go By | Frequency table distribution |

Motif Exclusion Parameters:

| | |
|---|---|
| Motifs to Eliminate | Patterns not selected |

RE Parameters:

| | |
|---|---|
| RE Sites to Eliminate | [NcoI, NdeI, BamHI] |
| RE Sites to Insert | |

Stem Loop Parameters:

| | |
|---|---|
| Eliminate Stem Loops | Yes |
| Minimum Loop Size | 9 |
| Maximum Stem Size | 6 |
| Percentage Mismatch Allowed | 0 |
| Eliminate Stem Loops with minimum | −8 |
| energy <= | |

Splice Site Parameters:

| | |
|---|---|
| Splice Site Model | Splice Site Model not selected |

Appended Tags Information:

| Appended tags information | NcoI AAGTGACCATGG (18) |
|---|---|
| | BamHI GGATCCCAATTG |
| | (2685) |

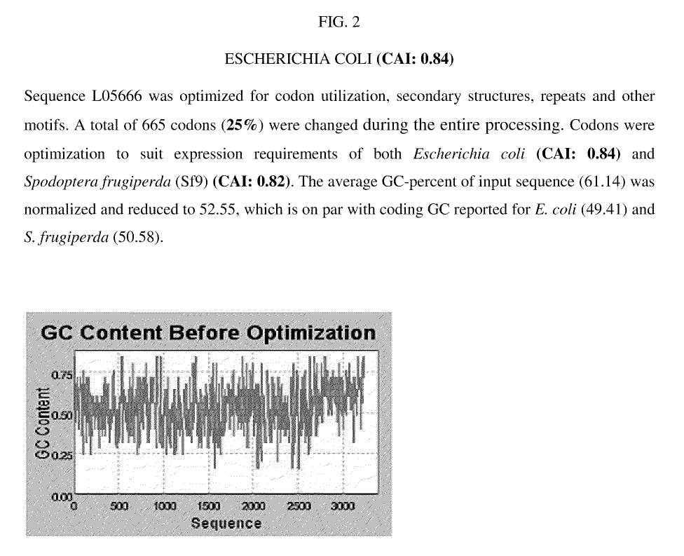

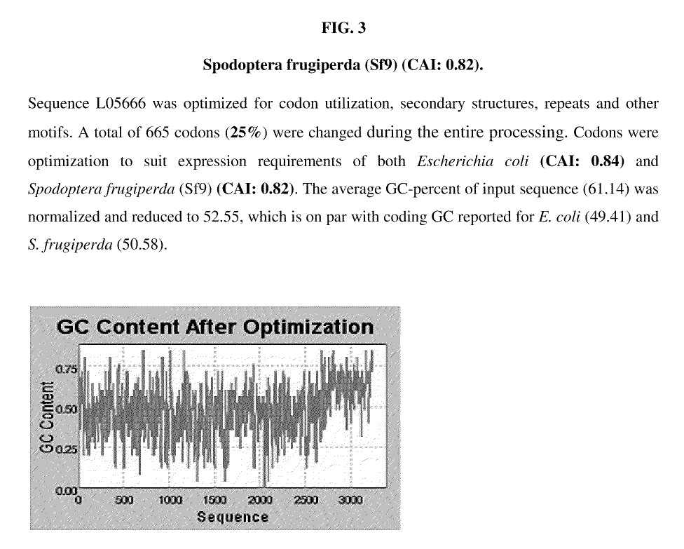

No Tags added. Codons were optimized to suit expression requirements of both Escherichia coli (CAI: 0.84) (Color Alteration Index) and Spodoptera frugiperda (Sf9) (CAI: 0.82). The average GC-percent of input sequence (61.14) was normalized and reduced to 52.55 (FIGS. 2-3), which is on par with coding GC reported for E. coli (49.41) and S. frugiperda (50.58).

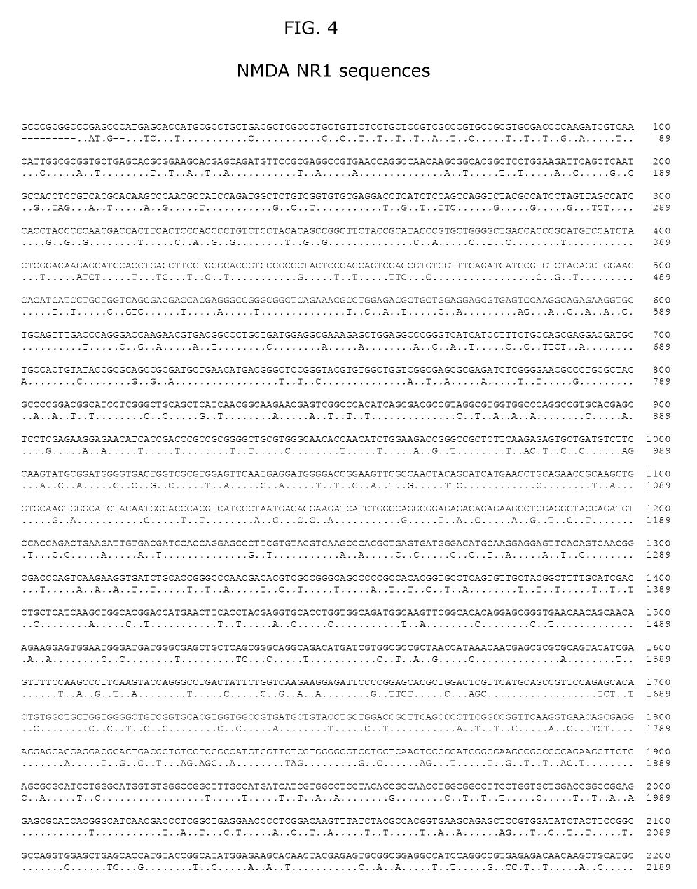

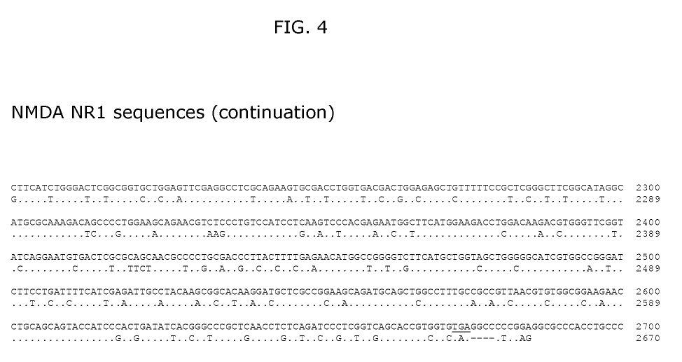

Restriction endonuclease (RE) Insert Sites: For cloning convenience, NcoI and XhoI restriction sites have been appended at ends and eliminated from the ORF. There were no changes in the amino acid sequence of L05666.1. Differences in the DNA sequences are indicated in FIG. 4 with L05666.1 at the top and the changes in the optimized sequence below.

In this study, we evaluated the feasibility of using DNA encoding NMDA-NR1 recombinant NR1 protein (r-NR1) or purified r-NR1 DNA encapsulated in PLGA microparticles to induce local and systemic NMDA-NR1-specific immunity following single-dose oral immunization.

### EXAMPLES

### Example 1

Plasmid Construction: To develop NMDA-NR1 DNA vaccine, the NR1 gene of NMDA was amplified by PCR and was cloned into the EcoRI/BamHI sites of plasmid vector pCMV-MCS (Invitrogen Life Technologies).

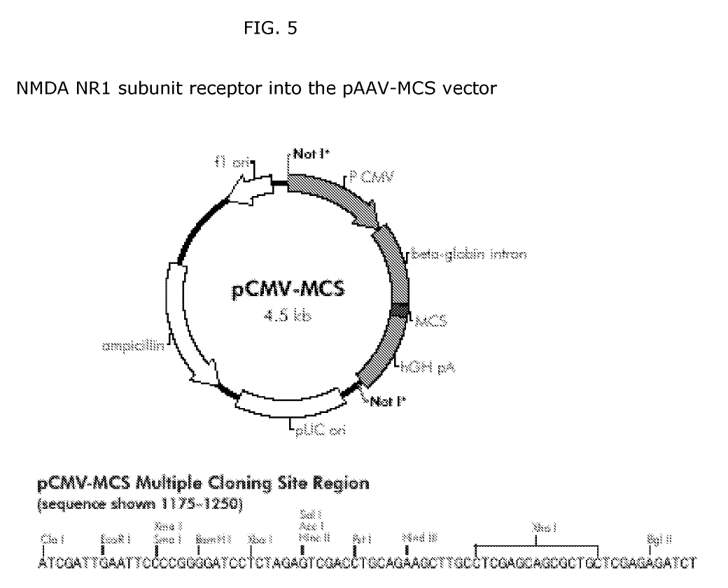

Cloning: Cloning of full length cDNA for the NR1 subunit of the N-methyl-D-aspartate (NMDA) receptor into the pAAV-MCS vector (FIG. 5)

Materials: The clone containing full length cDNA for NMDA was purchased from Invitrogen (Clone ID 19600412110003). The pAAV-MCS vector was purchased from Stratagene (Cat #240071). The vector contains the CMV promoter and other elements for high-level gene expression in mammalian cells when a gene of interest is cloned into the multiple-cloning site (MCS). The vector contains AAV-2 inverted terminal repeats (ITRs), which direct viral replication and packaging. The vector also contains pUC origin of replication (pUC ori) for propagation in E. coli to use as a DNA vaccine.

The primers were synthesized at Biosource as follows:

PCR primers:
NMDA-EcoRI-Forward:
ggggaattcATGAGCACCATGCGCCTGCTGACGCTCG
EcoRI
NMDA-BamHI-Reverse:
gggggatccTCACACCACGGTGCTGACCGAGGGATCT
BamHI
Sequencing primers:
Forward located at b-globin:
ATTCTGAGTCCAAGCTAGGC
Reverse located at hGH polyA:
TAGAAGGACACCTAGTCAGA

Methods: To clone into pAAV-MCS, the full length cDNA was amplified from the NMDA clone with the PCR primers appending EcoRI and BamHI sites. The PCR product and pAAV-MCS were digested with EcoRI and BamHI respectively to generate the cohesive ends. The NMDA gene then was ligated into pAAV-MCS and the vector was transformed into and propagated in DH5α for NR1 DNA propagation.

Results: The plasmid was prepared and the presence of the cloned insert was confirmed by DNA sequencing with the sequencing primers described above. The sequencing results generated with both forward and reverse sequencing primers were used to carry out a BLAST search of the NCBI database and aligned with NR1 subunit of the N-methyl-D-aspartate (NMDA) receptor (NCBI accession number L13266.1).

### Example 2

Baculovirus expression of NMDA-NR1 Recombinant protein (r-protein): Methods: To clone into baculovirus vector AcNMPV-1393, the full length cDNA was amplified from the NMDA clone with the PCR primers appending EcoRI and BamHI sites. The PCR product and pAAV-MCS were digested with EcoRI and BamHI to generate cohesive ends. The NMDA gene then was ligated into the baculovirus at the same restriction site and the vector was transformed and transfected into Spodoptera frugiperda (Sf-9) insect ovarian cells for the expression of recombinant (r) NMDA-NR1. NR1 genes were expressed in Baculovirus as per ours and others previously published methods (Kawamoto et al., 1995. Expression and characterization of the zeta 1 subunit of the N-methyl-D-aspartate (NMDA) receptor channel in a baculovirus system. Brain Res Mol Brain Res. 30:137-148; Lenhard et al., 1996. A new set of versatile vectors for the heterologous expression of foreign genes using the baculovirus system. Gene (Amst.) 169: 187-190; Sydow et al., 1996. Over expression of a functional NMDA receptor subunit (NMDAR1) in baculovirus-infected Trichoplusia ni insect cells. Brain Res Mol Brain Res. 41:228-40; Reddy et al., 1997. Application of recombinant bovine viral diarrhea virus proteins in the diagnosis of bovine viral diarrhea infection in cattle. Vet. Microbiol. 57:119-133 and Ivanovic et al., 1998. Expression and Initial Characterization of a Soluble Glycine Binding Domain of the N-Methyl-D-aspartate Receptor NR1 Subunit. J Biol Chem 273: 19933-19937).

The antigenicity of the baculovirus-expressed NR1 protein was detected by anti-NR1 specific monoclonal antibodies (Upstate Biotechnology, NY) in an enzyme-linked immunosorbent assay (ELISA), indirect immunofluorescent assay (IFA) and radio-immunoprecipitation (RIP).

The virus stocks were maintained at −70° C. in serum free Grace's medium. The proteins from recombinant NR1 baculovirus-infected insect cells were harvested and the supernatant was concentrated in a speed vacuum centrifuge. The isolated r-NR1 protein was analyzed by SDS-PAGE and stained with Coomassie blue.

The characterized virus was propagated, and infection of Sf9 cells with this recombinant baculovirus r-NR1 resulted in efficient production of the fusion protein. Five bands in the molecular mass range of 41-53 kDa were detected in infected cells and a prominent band of about 53 kDa was detected in the cell culture supernatant.

Purification of the NR1 (S1/S2) Protein: For purification of the heterologously-produced NMDA-NR1 protein, the cell culture supernatant of infected Sf-9 cells was harvested 3 days after infection with NR1 recombinant baculovirus. Further purification of the protein was performed using an anti-NR1 antibody-affinity column and the protein (53 kDa) was detected in RIP according to the methods described by Sydow et al., 1996 and Reddy et al., 1997 (see above). A soluble recombinant r-NR1 protein was derived from insect cell expression, confirming a previous report (Ivanovic et al., 1998. See above). Biochemical analysis of the produced protein revealed heterogeneity, because of N-linked glycosylation. Highly glycosylated protein was secreted into the insect cell medium. The glycosylation of NMDA receptor subunits in neurons is essential for correct targeting and subsequent secretion. Functional glycosylation of NR1 expressed in insect cells has been shown (Kawamoto et al., 1995; Ivanovic et al., 1998, see above). Immunoreactivity of r-NMDA-NR1 protein with NMDAR1 monoclonal antibodies proved that the baculovirus expressed NMDA-NR1 was properly folded and/or glycosylated in the insect cells. The recombinant protein was proved to be immunogenic in our animal stroke model experiments as well as having anti-MCAO and neuroprotective properties.

### Example 3

Preparation of PLGA: A Commonly Used Technique for Protein Encapsulation in microspheres was followed. Imperfect discharge and reduced stability of encapsulated proteins are common problems encountered when developing poly (lactide-co-glycolide) (PLGA) controlled-release systems. Antacid excipients in gastrointestinal fluid, which increase both microclimate pH and polymer water uptake, have been shown to prevent acid-induced instability of proteins encapsulated in PLGA.

The methodology of the present invention was also developed to validate these expectations. The in vitro r-NR1 protein release profile and stability of the released protein were examined, and the impact of the microencapsulation process on such properties was investigated. Encapsulation efficiency is an important quality of microcapsules, especially when used with costly therapeutics such as proteins. Another objective of this study was to optimize the methodology for efficient encapsulation.

Materials: Poly (lactic-co-glycolic acid) (PLGA; lactide:glycolide ratio (L:G)=50:50, intrinsic viscosity=0.59 dL/g, weight average molar mass (mw)=44 kDa). PLGA (L:G=50:50, intrinsic viscosity=0.15 dL/g, mw=13 kDa, Medisorb 5050DL2A).

### Example 4

Preparation of Microcapsules: Formulation of Cationic PLGA Nano-particles for in vitro GI Mucosa Studies: Preparation of microcapsules was done according to published methods (Yeo and Park, 2004. Characterization of Reservoir-Type Microcapsules Made by the Solvent Exchange Method. AAPS PharmSciTech 5:e52). Briefly, particles were prepared by a solvent extraction/precipitation method. PLGA (3 mg/ml) were dissolved in 2,2,2-Trifluoroethanol (TFE). 100 to 200 μg/mg microparticles NMDA-NR1 r-protein or 100-200 μg NMDA-NR1 DNA/mg microparticles in distilled water were added to the water/TFE mixture and stirred for 3 h on a magnetic stir plate to allow for TFE evaporation. The final formulation was 1% DNA to PLGA (w/w) and 5% r-protein to PLGA (w/w). The nanoparticle suspension was passed through a 1 μm Whatman syringe filter to remove large impurities and centrifuged for 75 min at 15,000×g at 4° C. to pellet the nanoparticles. Centrifugation conditions were carefully chosen so as to not pellet PLGA or DNA or protein particles. PLGA-DNA or r-protein nanoparticles were resuspended in distilled water and lyophilized or used directly for characterization or transport studies.

PLGA Nanoparticle Characterization: The size and surface morphology of the PLGA nano-particles were examined by scanning electron microscopy. The toxicity of PLGA-NR1 DNA or protein nanoparticles to porcine kidney (PK) cells was assayed using a propidium iodide nucleic acid stain (Molecular Probes, Eugene, Oreg.). PK cells seeded at 1×10⁶ cells/cm² were allowed to reach 60% confluence and then incubated with PLGA-NR1 DNA or protein nanoparticles. The naked DNA particles were harvested after 48 h. The PLGA-NR1 DNA or protein particle volume was adjusted to give final DNA concentrations of 1.0, 2.5, and 5.0 μg per well, and naked DNA or protein concentrations were adjusted to result in a final NR1 DNA or r-protein content of 2.5 μg and 10 μg per well. Harvested cells were resuspended in 500 μg/ml propidium iodide solution, which intercalates with DNA released from necrotic cells. No necrotic toxicity effect was observed with porcine cells with either PLGA or NMDA-NR1 DNA or r-protein. The percentage of dead cells in both control and test cultures was determined to be 13%, as assayed using flow cytometry. Adsorption and emission maxima for propidium iodide are 535 and 617 nm.

PLGA-NR1 DNA or r-protein nanoparticles with sizes less than 200 nm are designed to tightly bind DNA or protein with high efficiency. This small size is comparable to the acceptable size range for cationic particles that has been previously demonstrated to be effective (Tseng, 2001. Mechanics and multiple-particle tracking microheterogeneity of alpha-actinin-cross-linked actin filament networks. Biophys. J. 81:1643-1656).

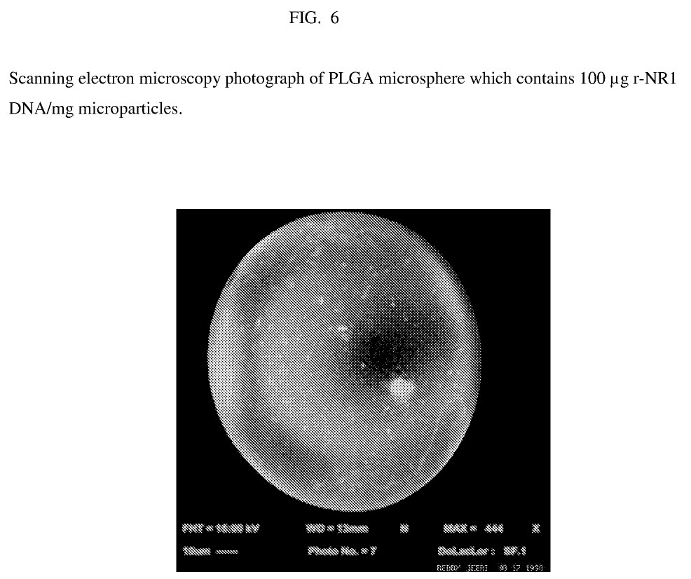

Microparticles were harvested by centrifugation, washed several times to remove the polyvinyl alcohol and residual solvent and finally lyophilized. A control formulation with the empty vector pAAV-MCS encapsulated into PLGA microparticles was similarly manufactured. The size and surface morphology of the PLGA nano-particles were examined by scanning electron microscopy. NR1 protein or DNA encapsulation ranged from 100 to 200 μg/mg microparticles which showed regular and spherical microsphere morphology, (FIGS. 6-7)

### Example 5

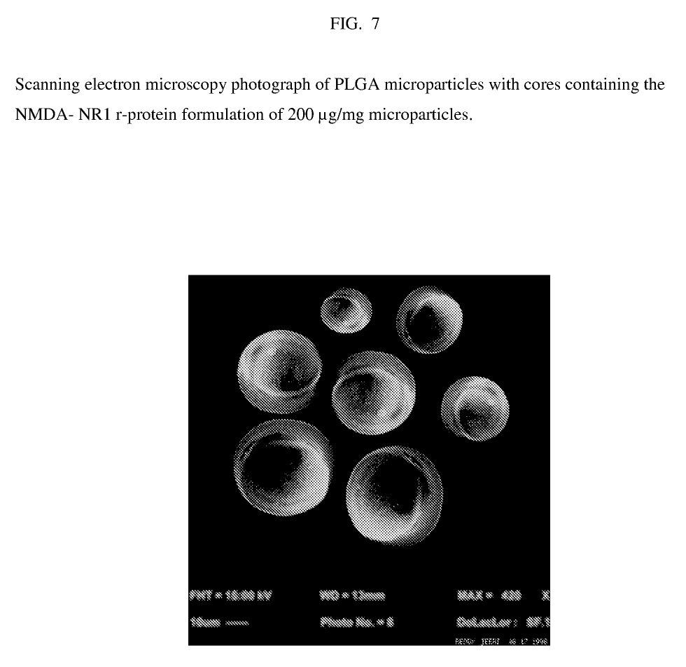

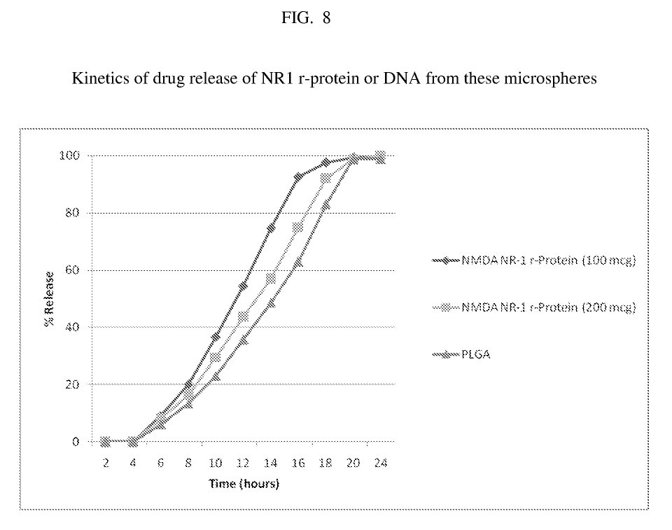

In Vitro Release of NMDA-NR1 Protein: The effect of PLGA on the NR1 protein release is shown in FIG. 8. A significant difference (P<0.05) in the rate and extent of drug release was observed in formulations with various amounts of protein and PLGA. The release of NR1 showed a biphasic pattern of drug release characterized by a burst release followed by a slow release which is characteristic of matrix diffusion kinetics (Lemoine et al., 1998. Preparation and characterization of alginate microspheres containing model antigen. Int J. Pharm. 176:9-19). The burst release can be reduced by increasing the polymer concentration resulting in better encapsulation efficiency.

Stability Studies: In view of the potential utility of the formulation for targeting rNR1 protein or DNA to the colon, the stability studies were performed at 39.5° C./75% RH for (climatic zone IV conditions for accelerating testing) to assess their long-term stability. After storage, the formulation was observed for physical appearance, particle size, particle shape, vaccine content and in vitro vaccine release studies.

Before and after storage at 39.5° C./75% RH for 6 months, in vitro release data were analyzed for dissolution efficiency as per Costa and Sousa Lobo (2001. Modelling and comparison of dissolution profiles. Eur J Pharm Sci. 13:123-133).

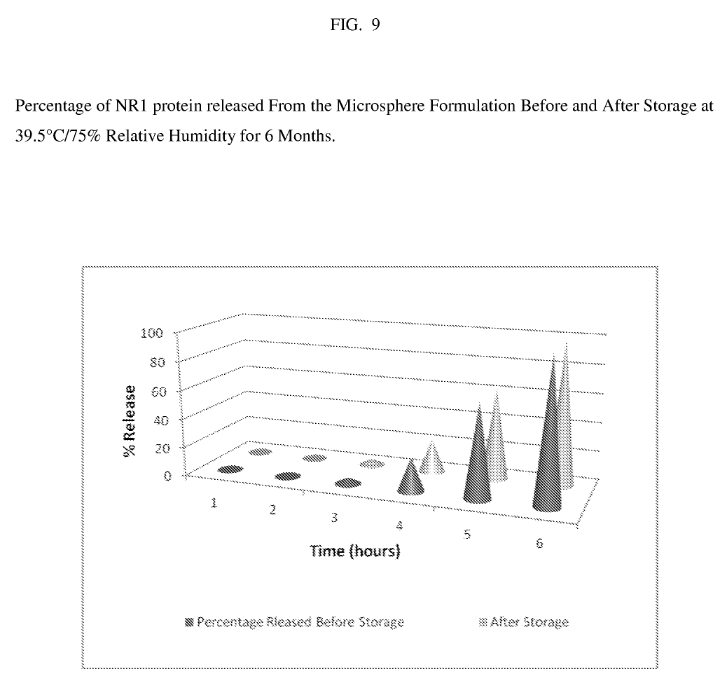

No significant difference (P>0.05) was found (FIG. 9). There was an insignificant change in the particle size distribution and shape indicating that the formulation of PLGA with r-NR1 protein or DNA could provide a minimum shelf life of 6 months.

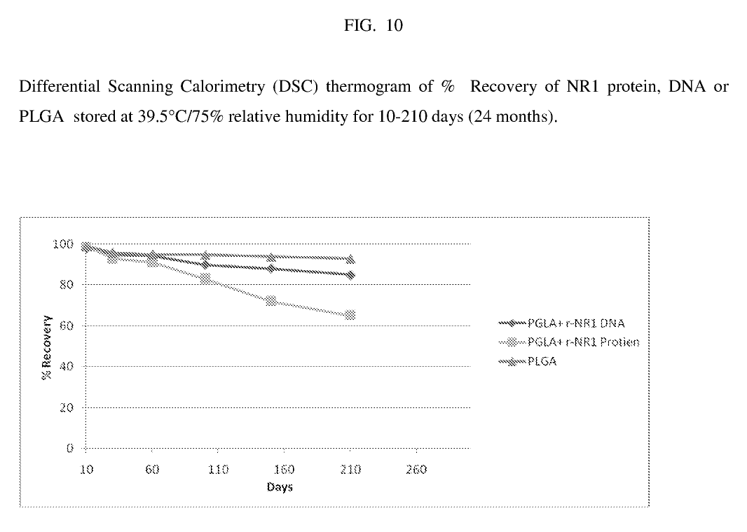

Thermal Characterization and Stability Studies: The thermal properties of PLGA microspheres were characterized to provide both qualitative and quantitative information about the physicochemical state of NR1 r-protein or DNA inside the PLGA microspheres. There is no detectable endotherm if the NR1 r-protein or DNA is present in a molecular dispersion or solid solution state within the polymeric PLGA microspheres. In the present investigation DSC thermograms were taken of pure NR1 r-protein or DNA, blank PLGA microspheres, NR1 r-protein or DNA-loaded within core PLGA microspheres, and NR1 r-protein or DNA and polymer physical mixtures in the same ratio as in the formulation. The prominent melting endotherms of the mixture of NR1 r-protein or DNA and polymer were found at 39.5° C. Thermal stability of PLGH, and NR1 DNA and NR1 r-protein encapsulated in PLGA was analyzed at 4-39.5° C. (FIG. 10)

Microsphere Characterization: Thermal analysis of blank PLGH polymer at 39.5° C. showed no degradation for the average particle size of the 3 lots. Incorporation efficiencies of 100% and 99% for NR1 r-protein or DNA revealed that most of the protein or DNA had been encapsulated within PLGA. Examination of the surface morphology of PLGA revealed that they were free of discernible surface pores.

PGLA Encapsulated NMDA-NR1 r-Protein Stability: The NR1 r-protein encapsulated in PGLA was substantially more stable up to 39.5° C.; 95% of the initial amount still remained after 180 days, 87% after 250 days, 71% after 300 days, and 63% after 305 days. Protein degradation was slower at lower temperatures in PBS, and as much as 97% of the initial amount still remained at 200 days at 2° C. and 4° C., respectively, in PBS. The NR1 protein was substantially more stable at pH 4.0 to 5.7, than at pH 7.4. The experiments conducted in the various pH conditions mimics the conditions which are likely to be encountered in the human gastrointestinal tract.

### Example 6

Transport of Polymeric Nanoparticle Gene Carriers in Gastric Mucus of Pigs: Cationic PLGA-microparticles which carry protein or DNA have been shown to adsorb to mucous surfaces and to efficiently transfect cells in vitro by several workers (Denis-Mize, et al., 2000. Plasmid DNA adsorbed onto cationic microparticles mediates target gene expression and antigen presentation by dendritic cells. Gene Ther. 7:2105-2112; Singh et al., 2001. Mucosal immunization with HIV-1 gag DNA on cationic microparticles prolongs gene expression and enhances local and systemic immunity. Vaccine 20:594-602). However, obtaining high transfection efficiencies in vivo is often limited by particle transport through extracellular barriers, including the mucosal barrier, which has been described as the foremost barrier to transfection in mucus-covered cells (Ferrari et al., 2001. Mucus altering agents as adjuncts for non-viral gene transfer to airway epithelium. Gene Ther. 8:1380-1386; Yonemitsu et al., 2000. Efficient gene transfer to airway epithelium using recombinant Sendai virus. Nat. Biotechnol. 18:970-973). To determine if cationic nanoparticles, formulated from PLGA with condensed protein or DNA, are effective gene carriers for administration to mucosal sites, we produced PLGA-protein/DNA nanoparticles and studied their transport rates in reconstituted pig gastric mucus (PGM) which had compositional and rheological properties physiologically relevant to natural gastrointestinal (GI) mucus (see below).

Reconstituted Pig Gastric Mucus (PGM): Mucus was formulated from 60 mg/ml PGM, 3.2 mg/ml DPPC, and 32 mg/ml BSA in sputum buffer (85 mM Na+, 75 mMCl—, 20 mM HEPES, pH 7.4; Sanders et al., 2000. Cystic fibrosis sputum: a barrier to the transport of nanospheres. Am J Respir Crit. Care Med. 162:1905-1911). Mucus was mixed on a stir plate for 48 h at 4° C. and stored at −20° C. as described. Reconstituted PGM had compositional and rheological properties physiologically relevant to gastrointestinal (GI) mucus (Khanvilkar et al., 2001. Drug transfer through mucus. K Adv Drug Deliv Rev. 48:173-193).

### Example 7

NR1-DNA or r-protein nano-particles embedded in PGLA: Following incubation in mucus, the potential measured for carboxylated-polystyrene (COOH—PS) particles (−17.5 mV at pH 6) was close to that for PLGA-NR1 DNA or r-protein nanoparticles (−11+/−7 mV at pH 6), even though the initial potentials of the two particle types were quite different prior to incubation with mucus (39+/−6 for PLGA-DDAB/DNA and −5 mV for COOH—PS). The mobility of COOH—PS and PLGA-NR1 DNA or r-protein nanoparticles in PGM was tracked in real time in two dimensions and the results suggest that mucus components readily adsorb to the surface of each type of particle within minutes of their addition to mucus.

Nanoparticle transport through mucosal barriers is often restricted because of mucoadhesion and the highly viscoelastic nature of mucus gels, which may limit efficient drug and gene delivery. We formulated particulates smaller than 200 nm from PLGA. Using the method of multiple-particle tracking (MPT) we measured the transport rates of dozens of individual PLGA-NR1 DNA or r-protein nano-particles in real time in reconstituted pig gastric mucus (PGM). The average transport rate of PLGA-NR1 DNA or -r-protein nano-particles was 10-fold higher than those of similar size polystyrene nano-particles.

### Example 8

In Vivo Swine Model Study: We chose the swine model to test NMDA-NR1 protection against stroke because pigs are similar to humans in the function and relative size of many internal organs.

We investigated the pH of the gastrointestinal tract of the pigs, which includes the stomach, small intestine, cecum and large intestine, prior to immunization and found that in all experimental pigs the overall gastric pH was about 4.0 without feed. The pH of the small intestine is 6-7, which is higher than that of the other organs of the GI perhaps because of pancreatic secretions and lower acid production. The pH of the cecum and the large intestine are somewhat lower (about 5) than the small intestine because of the activity of anaerobic bacteria that break down carbohydrates and produce volatile fatty acids. Food or water can temporarily alter pH levels by buffering against agents which may naturally occur in the feed components and because they dilute gastric juices. A change of diet and/or lack of feed consumption at weaning can also alter pH to around 4.0.

Sixty certified pathogen-free 12-week-old male and female pigs (approximately 88 to 150 lbs) were obtained from South Dakota farms. They were assigned to 5 groups of 12 pigs and each group was housed in separate pens per treatment. Animals were treated in compliance with the regulations on Animal Care under protocols approved by the Committee on Animal Care and Supply.

Allocation of animals per group:

Treatment 1: NMDA-NR1 r-protein=12 pigs;
Treatment 2: NMDA-NR1 DNA=12 pigs;
Treatment 3: PLGA=12 pigs;
Treatment 4: Vector=12 pigs;
Treatment 5: PBS=12 pigs.

Immunization Preparation: The animals were fed a standard diet and maintained on a 12 h light/dark cycle (lights on 6:00 A.M. to 6:00 P.M.). All experimental procedures were approved by the Institutional Animal Care and Use Committee.

### Example 9

In vivo Stomach and Gastro Intestine Preparation and Immunization: To prepare the pigs for in vivo vaccination with PLGA-encapsulated NMDA-NR1 r-protein or DNA delivered to the mucosal surface of the pigs, the pH of the gastro intestine (GI) of experimental pigs was measured for fed animals and fasted animals. The pH of the fed animal GI was 5.1, and for fasted animals, the luminal pH was 4 to 4.2 which mimicked the known fasted-to-fed transition of intragastric pH in pigs. Acidic gel secreted through the mucus contributed to the intragastric pH of the fasted, but not fed, animal. To permit effective vaccine absorption in the GI of the animals, pigs were fasted overnight prior to beginning the experiments. All animals were then housed in a barn that permitted them access to water and to feed.

A single dose of vaccine was given to fasted animals through oral administration with NMDA-NR1 r-protein or DNA encapsulated into PLGA microparticles using a 24-gauge feeding needle with a capillary tube (24 cm). The microparticles contained a dose of either 100 or 200 μg NMDA r-protein encapsulated with PLGA or 100 or 200 μg DNA encapsulated with PLGA (pre-dose determined) suspended in 10 ml per pig, which was administered to fasted animals (6 pigs per dose; total 12 per treatment). The control groups received the same amounts of a placebo which consisted of plain PLGA microparticles (100 or 200 μg) or pAAV-MCS (100 or 200 μg) (6 pigs per dose; total 12 per treatment) or PBS by the same route (12 pigs).

Blood samples were collected by jugular or anterior vena cava venipuncture with evacuated serum tubes and heparin tubes for blood analysis. Serum samples were taken before the experiment, and at 0, 4, 8 and 24 weeks after immunization and were tested for the presence of anti-NMDA-NR1 r-protein or anti-NR1 DNA antibodies. The collected sera were stored at −80° C. until analyzed. Fecal washes were prepared from 2 g fecal samples in 20 ml PBS, incubated at 30° C. for 30 min and centrifuged at 10,000×g for 10 min. The supernatants, which contained anti-NMDA-NR1 IgA antibodies, were stored at −80° C. for further analysis.

Nasal secretions were collected with 2 absorbent swabs after 150 μL of phosphate buffered saline (PBS) (pH 7.3) was sprayed into each nostril. The swabs were placed proximal to the external nares to absorb fluid without disrupting the nasal mucosa. The nasal collections were centrifuged for 30 seconds at 10,000×g. Nasal swabs were placed in 1.5-ml Eppendorf tubes and stored at −80° C.

### Example 10

Preventive vaccine for stroke: Detection of antigen-specific antibodies: The concentrations of NMDA-NR1-specific antibodies in serum, nasal secretions, and fecal samples were determined by an enzyme-linked immunosorbent assay (ELISA). Ninety-six well plates were coated with 0.1 μg of antigen per well and incubated with serially diluted samples. Murine anti-porcine immunoglobulin A (IgA) 1/250, IgG1 1/200, or IgG2 1/500, followed by biotinylated-goat anti-mouse IgG (H+L) 1/5000 or alkaline phosphatase-goat anti-porcine IgG (H+L) 1/5000, were used as detecting antibodies. Di-(Tris) p-nitrophenyl-phosphate was used as the chromogenic substrate. A reference standard was prepared using a porcine immunoglobulin reference serum, in which 1 unit is equivalent to 1 μg. Standards of 1.95 to 1000 ng/ml and 0.78 to 100 ng/ml were used to estimate IgG and IgA concentrations, respectively, in serum, nasal or fecal samples.

Clinical Observations and Sampling: Clinical signs and body temperature of all pigs were monitored and oro-pharyngeal fluid was collected daily for 8 days following vaccination with NMDA-NR1 r-protein or DNA PGLA microparticles. The following clinical signs were scored: labored breathing, abdominal breathing, anorexia, apathy and coughing. Blood was collected at a Proportional-Integral-Derivative (PID) 0, 4, 8, and 24 weeks. Heparinized blood was collected for the isolation of peripheral blood mononuclear cells (PBMCs) to be used in a T-cell proliferation assay. Clinical observations were made during 2 days after oral immunization, the body temperatures and clinical responses of the pigs were recorded at 3-h intervals. Each animal received a daily average score. Scores of 0 (normal), 1 (slight), 2 (marked), or 3 (severe) were based upon the increase in respiratory rate, respiratory effort, presence of anorexia, and degree of lethargy.

Statistical Analysis: The Kruskal-Wallis non-parametric rank sum test and Wilcoxon's matched pairs test were used to test the significance of differences in humoral responses. Analysis of variance (ANOVA) was also performed. Data were analyzed using SAS 8.2 (SAS Institution Inc.). A p value<0.05 was considered significant.

Humoral Immune Response: NMDA-NR1-specific total antibody from serum IgG and intestinal IgA were analyzed by ELISA in serum or fecal supernatants and nasal swabs from individual pigs in each group with previously standardized NMDA-NR1 protein-coated ELISA plates.

The solid phase was incubated at 4° C. with NMDA-NR1-specific antibodies followed by incubation with antibodies specific for pig IgA or IgG. Serum from animals immunized with PGLA microparticles was used as the negative control (blanks). Total antibody in serum was measured with a serially-diluted standard antibody, as described above. Mean Optical Density (OD) value for sera from animals immunized with 100 and 200 μg NMDA NR-1 protein, or pAAV-NR1 DNA was about 1.2 in contrast with the control animal sera (pAAV, PLGA) for which O.D. was about 0.4 (Table 1).

Cell Mediated Immune Response: CMI responses were measured by the procedures described earlier (Furesz, et al. 1997. Antibody and cell-mediated immune responses of Actinobacillus pleuropneumoniae-infected and bacterin-vaccinated pigs. Infect Immun. 65:358-365 and Reddy et al., 1999. Semiliki forest virus vector carrying the bovine viral diarrhea virus NS3 (p80) cDNA induced immune responses in mice and expressed BVDV protein in mammalian cells. Com Imm Micro Infec Dis. 22: 31-246).

Statistical Analysis Tests for significant correlation between compared treatments were carried out using the Mantel-Haenszel correlation test. A significance level of a=0.05 was used for all tests. The Goodman and Kruskalis g-statistics were used to measure the correlation between the treatments. All analyses were carried out using the SAS ‘FREQ’ procedures Landis J R, Miller M E, Davis C S, Koch G G. Department of Biostatistics, School of Public Health, University of Michigan, Ann Arbor 48109.

Preventive Vaccine for Stroke: Oral Immunization of NMDA-NR1 Induces Humoral Immunity in Pigs: Pigs immunized with NMDA-NR1 r-protein or DNA developed an increase in antibody concentrations to r-NR1. In ELISA, the average optical density (OD) value for NMDA-NR1 r-protein or DNA was 1.1 compared with the pAAV-MCS control which gave an OD of 0.4. The NMDA-NR1 r-protein or DNA dose dependent-antibody responses are also presented in Table 1.

The pre-immune sera from the non-inoculated animals or PBS inoculated animals, as well as from control pigs inoculated with the identical plasmid backbone or PLGA particles, failed to show reactivity. The sera from pigs inoculated with the NMDA-NR1 r-protein or DNA construct reacted with recombinant r-NR1 protein as determined by indirect immunofluorescent assay. The pre-immune sera and sera from non-vaccinated animals or from control pigs inoculated with the pAAV-MCS plasmid without NR1 insert failed to show reactivity. The immune responses of all groups of animals were consistent. A single inoculation of 100-200 μg of r-NR1 protein or NR1 DNA (which expresses NMDA-NR1) encapsulated in PLGA resulted in the seroconversion of 100% of the animals after inoculation.

TABLE
NR1 specific IgG antibody responses in pig sera after NMDA-NR1 r-
protein or DNA oral immunization

| | | | | | |
|---|---|---|---|---|---|
| Immunogen | dose | 0-week | 4-week | 8-week | 24-week |

IgG μg/ml (mean ± SD)

| | | | | | |
|---|---|---|---|---|---|
| NR1 r-P | (100 μg) | 017 ± 50 | 185 ± 47 | 210 ± 50 | 280 ± 100 |
| NR1 r-P | (200 μg) | 017 ± 50 | 180 ± 50 | 210 ± 50 | 280 ± 100 |
| pAAV-NR1 | (100 μg) | 017 ± 50 | 155 ± 47 | 190 ± 50 | 210 ± 100 |
| pAAV-NR1 | (200 μg) | 017 ± 50 | 300 ± 50 | 345 ± 50 | 500 ± 100 |
| pAAV-MCS | (100 μg) | 017 ± 50 | 035 ± 47 | 048 ± 50 | 060 ± 100 |
| pAAV-MCS | (200 μg) | 017 ± 50 | 035 ± 47 | 050 ± 50 | 060 ± 100 |
| PLGA | (100 μg) | 017 ± 50 | 025 ± 47 | 038 ± 50 | 050 ± 100 |
| PLGA | (200 μg) | 017 ± 50 | 025 ± 47 | 030 ± 50 | 050 ± 100 |
| PBS | — | 017 ± 50 | 017 ± 50 | 017 ± 50 | 017 ± 100 |

Production of NR1-specific IgG antibodies in pigs immunized with NMDA-NR1 r-protein or DNA or plasmid pAAV-MCS or PBS: Blood was drawn at 0, 4, 8, and 24 weeks. Anti-NR1 antibodies in two-fold diluted serum from each group was assayed by an ELISA with NR1 purified r-protein from Sf-9 insect cells. The results are the mean ELISA titer±SD detected at each time point after immunization. Pigs immunized with blank pAAV-MCS, PLGA or PBS generated nonspecific or normal pig antibodies.

TABLE
Fecal IgA

| | | | |
|---|---|---|---|
| 0-week | 4-week | 8-week | 24-week |

| | |
|---|---|
| Immunogen | μg/mg IgA |

| | | | | |
|---|---|---|---|---|
| NR1 r-P | 30.00 | 40.00 | 60.00 | 70.00 |
| pAAV-NR1 | 20.00 | 30.00 | 40.00 | 60.00 |
| pAAV-MCS | 20.00 | 20.00 | 30.00 | 40.00 |
| PLGA | 20.00 | 20.00 | 30.00 | 40.00 |
| PBS | 23.00 | 21.00 | 22.00 | 23.00 |

TABLE
Nasal IgA

| | | | |
|---|---|---|---|
| 0-week | 4-week | 8-week | 24-week |

| | |
|---|---|
| Immunogen | μg/mg IgA |

| | | | | |
|---|---|---|---|---|
| NR1 r-P | 35.00 | 38.00 | 40.00 | 45.00 |
| pAAV-NR1 | 20.00 | 30.00 | 30.00 | 35.00 |
| pAAV-MCS | 20.00 | 20.00 | 30.00 | 30.00 |
| PLGA | 20.00 | 25.00 | 23.00 | 26.00 |
| PBS | 20.00 | 21.00 | 22.00 | 23.00 |

Levels of IgA antibodies to NMDA-NR1 r-protein: Production of NR1-specific IgA antibodies in pigs immunized with NMDA-NR1 r-protein or DNA or control plasmid pAAV-MCS or PBS. Samples were collected at 0, 4, 8, and 24 weeks. Anti-NR1 antibodies in two-fold diluted pooled serum from each group (12 pigs) were assayed by an ELISA with purified NR1 r-protein.

Cell Mediated Immune Response: There were no detectable cell mediated immune responses after vaccination with NMDA-NR1 r-protein or DNA as measured by the procedures described earlier (Reddy et al., 1999. Semiliki forest virus vector carrying the bovine viral diarrhea virus NS3 (p80) cDNA induced immune responses in mice and expressed BVDV protein in mammalian cells. Com Imm Micro Infec Dis. 22: 31-246).

Clinical Scores: All animals receiving the treatments showed clinical scores less than 1 (Scores of 0 (normal), 1 (slight), 2 (marked), or 3 (severe) were based upon the increase in respiratory rate, respiratory effort, presence of anorexia, and degree of lethargy) and body temperatures ranged from 37-38.5° C. No abnormal clinical scores or temperatures were observed before or after immunization with NMDA-NR1 r-protein or DNA at 0, 4, 8, 24 weeks. The animals were kept in pens for 6-months.

### Example 11

Experimental Stroke to detect the vaccine efficacy for prevention of Stroke: Methods: Awake pigs were induced for middle cerebral artery occlusion (MCAO) by peri-vascular microinjection of endothelin-1 (ET-1). Endothelin-1 (Nova Biochem) was dissolved in sterile saline and was injected (60 pmol in 3 μl) via a 31-gauge guide cannula stereotaxically placed 0.5 mm above the middle cerebral artery below the dura. The cannula was left in situ for 5 min before slowly being withdrawn over 2-3 min. Functional outcome was determined 0, 30, 60 and 80 minutes after stroke by neurological deficit score, motor performance, and sensory hemi-neglect tests. Fifty-percent of the pigs were sacrificed at 80 min, and infarct area and volume were determined by histology and computerized image analysis.

Oral Vaccination with NMDA-NR1 r-protein or DNA: Endothelin-1-induced MCAO resulted in marked reduction of functional deficits and neuronal damage in vaccinated animals. A significant reduction of cortical damage was observed in response to treatment with NMDA-NR1 r-protein or with NMDA-NR1 DNA. In addition to cortical protection, NMDA-NR1 r-protein or DNA induced full-scale protection from neuronal damage and stroke. The functional outcome paralleled the histopathology. Rota-rod performance, sensory hemi-neglect, and neurological deficit scores returned to pre-ischemia levels in pigs orally treated with NMDA-NR1 r-protein or DNA PLGA-encapsulated particles.

### Results

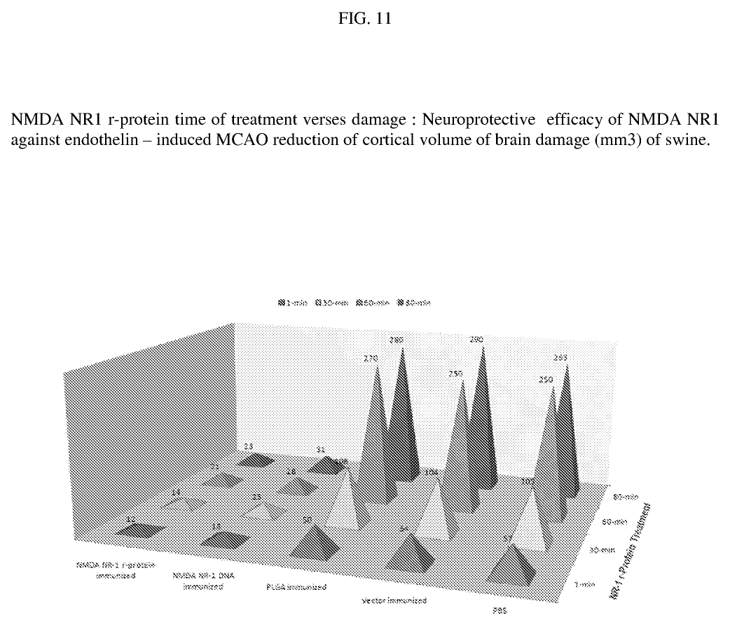

Animals vaccinated with NMDA-NR1 r-protein or DNA showed minimal physiological effects of Endothelin-1-induced MCAO, recovered fully in 80 min and survived without symptoms of stroke in a pen until the end of the study. The damage induced by ET-1-induced MCAO is presented in FIG. 11 and compared to non-vaccinated animals treated with intravenous NMDA-NR1 r-protein. In non-vaccinated animals both cortical and striatal damage was significantly reduced by treatment with NMDA-NR1 protein within 30 minutes after stroke (FIG. 11).

The vaccinated animals were immune to ET-1 induced MCAO and showed minimal symptoms. Additional protection from the NMDA-NR1 protein treatment was not statistically significant. However, the present findings suggest that immunization with NMDA-NR1 protein or DNA in PLGA-microparticles may have great promise in the treatment of human stroke.

Pigs expressing NR1 DNA or protein antigen-presenting cells in the mucosa of the GI epithelium are protected from ischemia. Studies have shown that the inhibition of lipid peroxidation attenuates neuronal damage in animal models of cerebral ischemia and reduces brain infarct volume in a MCAO model of stroke, when NR1 was administered to animals which were not vaccinated with NR1 (During, et al., 2000. An oral vaccine against NMDAR1 with efficacy in experimental stroke and epilepsy. Science 287:1453-1460). This latter result suggests a mechanism for the therapeutic properties of the NR1 subunits of NMDA after ischemia.

In addition, blockade of Na+ channels has been proposed as a possible neuroprotective mechanism by reducing energy expenditure in compromised tissue (Adkins et. al., 2004. behavioral and neuro plastic effects of focal endothelin-1 induced sensori motor cortex lesions. Neuroscience 128: 473-486; Urenjak and Obrenovitch 1996. Pharmacological modulation of voltage-gated Na+ channels: a rational and effective strategy against ischaemic brain damage. Pharmacol Rev. 48:21-67; Callaway et al., 1999. Delayed Treatment With AM-36, a Novel Neuroprotective Agent, Reduces Neuronal Damage After Endothelin-1-Induced Middle Cerebral Artery Occlusion in Conscious Rats. Stroke. 30:2704-2712). A large part of the energy expenditure of excitable cells is used to maintain Na+ and K+ gradients across cell membranes, which indicates that the blockade of Na+ channels may constitute an effective neuroprotective mechanism. The results of a number of studies strongly suggest that the administration of inhibitors of voltage-sensitive Na+ channels is beneficial, even when it is delayed after stroke. A number of structurally unrelated neuroprotective drugs have been shown to act by down-modulating Na+ channels, suggesting that this action may constitute an important mechanism. Compounds which act by Na+ channel blocking have been shown to be neuroprotective in MCAO models of ischemia.

There is very often a delay in the time taken to hospitalize and diagnose a stroke victim, and it is now considered important to demonstrate significant neuroprotection for treatments administrated after ET-1 induced stroke. Hence, the purpose of the present study was to determine the effectiveness of the novel neuroprotective agent NMDA-NR1 administered at various intervals after the onset of stroke. This study used the endothelin-1 (ET-1) model of MCAO, which is less invasive than some other models, incorporates reperfusion, and has the advantage that the pigs are conscious during the stroke. Our present results indicate that NMDA-NR1 potently protects against neuronal damage when administered within 30 minutes and improves the functional outcome for recovery at 24 weeks.

Therapeutic Properties of NMDA-NR1 r-protein for stroke induced in non-vaccinated animals: The control animals in which a stroke was induced by endothelin-1 were treated intravenously with NMDA-NR1 protein at 100 μmol/lb animal weight/25 lb [not encapsulated in PLGA-microparticles]. [NMDA-NR1 protein 100 μmol/lb animal weight/25 lb was predetermined by another independent experiment where 10-500 μmol was tried and 100 μmol was found to be the appropriate quantity with minimum toxicity to the animal (data not shown). Interestingly, neuronal damage was reduced, with the greatest protection found with NMDA-NR1 protein treatment but not with NMDA-NR1 DNA treatment administered within 0 to 30 minutes after stroke in animals that were not vaccinated with NMDA-NR1 r-protein or DNA. Striatal damage was significantly reduced after treatment with NMDA-NR1 protein 0 to 30-minutes after stroke, but damage increased with delayed treatment (FIG. 11). The functional outcome paralleled the histopathology. Rota-rod performance, sensory hemi-neglect, and neurological deficit scores (not shown) returned to pre-ischemia levels in pigs treated intravenously with non PLGA encapsulated NMDA-NR1 r-protein (FIG. 11). Damage to the frontal cortex and dorsal lateral striatum was observed with delayed treatment at 60 and 80 minutes in unvaccinated animals

Effect of treatment delay in unvaccinated animals: The greatest protection against stroke was found in unvaccinated and treated animals where treatment was delayed by 0 minutes after stroke. The damage to the frontal cortex and dorsal lateral striatum increased with delayed treatment 30, 60 and 80 minutes in unvaccinated animals.

Clinical Scores Neurological abnormalities were evaluated using a neurological deficit score based on detection of abnormal posture and hemiplegia, as described by De Ryck et al. (1989. Photochemical stroke model: flunarizine prevents sensorimotor deficits after neocortical infarcts in rats. Stroke. 20:1383-1390). Abnormal posture was assessed by suspending pigs by the tail and observing twisting of the thorax and extension of forelimbs. The presence of thorax twisting and the absence of contra lateral forepaw extension were scored at 1 each. Hemiplegia was evaluated by placing pigs on a raised platform. When there is a deficit the contra lateral hind limb slips off the edge of the platform (score=1), and the contra lateral forelimb slips off when the snout and whiskers lose contact with the surface (score=1). Thus, when the scores were summed, the maximum neurological deficit score was 4. A score of 0 was considered normal.

Motor impairment was assessed with the use of the accelerating rota-rod (Ugo Basile, model 7750). Pigs were given two training sessions, 10 minutes apart, before surgery. Tendency to fall off the rota-rod was then determined before ET-1 induction MACO and after stroke. Pigs not falling off within 5 minutes were given a maximum score of 300 seconds.

Sensory hemi-neglect was evaluated by a test developed by Schallert and Whishaw (1984. Bilateral cutaneous stimulation of the somatosensory system in hemidecorticate rats. Behav Neurosci. 98:518-540) that measures sensitivity to simultaneous forelimb stimulation. This test is based on observations of behavior in humans with unilateral brain damage (Daffner et. al., 1990. Dissociated neglect behavior following sequential strokes in the right hemisphere. Ann Neurol. 28:97-101). If two stimuli are presented simultaneously, one on each side of the body, the contra lateral stimulus appears to be masked (“extinguished”), and either remains undetected until the ipsilateral stimulus is removed or feels subjectively weaker. In pigs, the test consists of placing adhesive tapes (Avery adhesive labels, 1-cm circles) on the distal-radial region of each wrist. Placement of the first tape was randomized between contra lateral and ipsilateral limbs. The tape on both forepaws was touched simultaneously before the animal was placed in a pen of the pig barn. Removal of the tape by the animals was measured by spontaneous activity of individual pigs was measured for 10 minutes at the same time each day with an Animex type S activity meter (FARAD Electronics). Pigs were equilibrated presurgery in the apparatus for a period of 30 minutes before the first testing time.

Sensory hemi-neglect was evaluated by a test that measures sensitivity to simultaneous forelimb stimulation (Schallert and Whishaw. 1984. Bilateral cutaneous stimulation of the somatosensory system in hemi-decorticated pigs. Behav. Neurosci. 98:518-540). This test is based on observations of behavior in humans with unilateral brain damage (Daffner et al., 1990. Dissociated neglect behaviour following sequential strokes in the right hemisphere. Ann Neurol. 28:97-101; Ferro J M, Kertesz and Black 1987. Subcortical neglect: quantitation, anatomy, and recovery. Neurology. 37:1487-1492). If 2 stimuli are presented simultaneously, 1 on each side of the body, the contralateral stimulus appears to be masked (“extinguished”), and either remains undetected until the ipsilateral stimulus is removed or feels subjectively weaker. In pigs, the test consists of placing adhesive tapes on the distal-radial region of each wrist. Placement of the first tape was randomized between contralateral and ipsilateral limbs. The tape on both forepaws was touched simultaneously before the animal was placed in a Plexiglas cage, and both the latency to touch and the latency to remove each stimulus from the contralateral and ipsilateral forepaws were measured with a stopwatch. The test was terminated at 120 seconds if the tapes had not already been removed.

Latency to touch and remove an adhesive tape from the contralateral forepaw was consistently and significantly increased in control vehicle-treated pigs. In comparison, there was no change compared with before stroke in the time taken to touch or remove a tape simultaneously placed on the ipsilateral forepaw. If the NR1 protein treatment was given to unvaccinated pigs either 1 or 30 minutes after stroke, the difference in latency between contralateral and ipsilateral forepaws was reduced compared to untreated pigs, and the latency to touch and remove tapes returned to presurgery times by 80 hours after stroke. Whereas, if the NR1 dose was delayed until 60 or 80 minutes after stroke, the difference in latency between contralateral and ipsilateral forepaws was not reduced and did not recover even after 80 hours after stroke induced by ET-1.

Histopathological Assessment of Brain Damage: Induction of Focal Cerebral Ischemia: Unvaccinated Pigs treated with NMDA-NR1 r-protein were anesthetized with halothane (4% for induction; 1-2% for maintenance) in nitrous oxide/oxygen (80/20%; v/v). Normothermia (37±1° C.) was maintained by using a thermostatically-controlled heating blanket connected to a rectal thermometer. Endothelin-1 (60 pmol in 3 μl) was injected via a 31-gauge guide cannula stereotaxically placed 0.5 mm above the middle cerebral artery (AP+0.2 mm; ML . . . 5.9 mm; DV . . . 7.0 mm below dura). The cannula was left in situ for 5 min before slowly being withdrawn over 2-3 min. Animals were placed in an incubator to maintain normothermia until their recovery from anesthesia.

Pigs were re-anesthetized with pentobarbitone (Sagittal; 60 mg/kg) 80-minutes after injection of ET-1 and brains were fixed by transcardiac perfusion first with 20 ml of heparinized saline (10 U/ml), followed by 200 ml of 4% paraformaldehyde in 50 mM PBS, pH 7.4. The brain was removed intact and immersed in fixative containing 10% sucrose for at least 24 hr before cryostat sectioning. Coronal sections (20 μm thick) were cut and stained with either cresyl violet or thionine. The volume of brain damage was determined as described previously (Park et al., 1989. Effect of the NMDA antagonist MK-801 on local cerebral blood flow in focal cerebral ischaemia in the rat. J Cereb Blood Flow Metab. 9:617-622; Sharkey and Butcher, 1994. Immunophilins mediate the neuroprotective effects of FK506 in focal cerebral ischaemia. Nature. 371:336-9). Briefly, the area of brain damage of eight selected brains was assessed using light microscopy by an observer who was unaware of the treatment groups. The volume of brain damage was calculated by integration of the cross-sectional area of damage at each stereotaxic level and the distances between the various levels (Park et al., 1989; Sharkey and Butcher, 1994, see above).

All procedures used in this study were performed in accordance with the Prevention of Cruelty to Animals Act. A 23-gauge stainless steel guide cannula was stereotaxically implanted into the piriform cortex 2 mm dorsal to the right MCA according to the method of Sharkey et al. (1993. Perivascular microapplication of endothelin-1: a new model of focal cerebral ischemia in the rat. J Cereb Blood Flow Metab. 13:865-871). The stereotaxic coordinates were modified (0.2 mm anterior, −5.2 mm lateral, and −6.1 mm ventral, according to a stereotaxic atlas (De Ryck et al., 1989. Photochemical stroke model: flunarizine prevents sensorimotor deficits after neocortical infarcts in rats. Stroke. 20:1383-1390). The cannula was secured with dental acrylate cement, and 2 small screws were inserted into the skull. The scalp was closed with sutures. The animals treated with NMDA-NR1 protein were housed individually and allowed to recover for 80-hours.

Necropsy of Experimental Pigs and Quantification of Ischemic Damage: Pig brains were removed and frozen in liquid nitrogen and stored at −80° C. Coronal cryostat sections (18 μm thick) were cut at 8 predetermined planes throughout the brain from −3.2 to 6.8 mm anterior to the interaural line. The sections were then fixed in paraformaldehyde vapor for 30 minutes at 60° C. and then overnight at room temperature before they were stored at −80° C. Infarct area was measured in unstained sections, which employs an image analysis system (MCID M4 image analyzer, Imaging Research Inc) to trace the areas of damage in each brain section. Total infarct volume was calculated by integrating the cross-sectional area of damage at each stereotaxic level and the distances between the levels according to the method of Osborne et al. (1987. Quantitative assessment of early brain damage in a rat model of focal cerebral ischaemia. J Neurol Neurosurg Psychiatry. 50:402-410).

The infarct area in the cortex was significantly reduced at several stereotaxic levels in NMDA-NR1 r-protein or DNA vaccinated animals as well as in NMDA-NR1 protein treated animals. When administration of NMDA-NR1 protein was delayed by 1 or 30 minutes after MCAO NMDA-NR1 r-protein significantly reduced the damage in the striatum in non-vaccinated and with minimal damage among vaccinated group. There was a significant linear trend for the degree of protection, which increased as the time delay in drug administration decreased (P=0.02). The volume of excitotoxic brain damage in the cortex induced by Endothelin-1 in control animals (PLGA, Vector or PBS) after intravenous treatment with NMDA-NR1 r-protein at 100 μmol/lb weight (25 lb) of the animal was 30% of the cortex at O-hours, 60% at 30-minutes, 65% at 60-minutes and almost 80-90% at 80-minutes. The delay in treatment with NMDA-NR1 r-protein increased the damage of cortex. Data are the mean volume of brain damage for groups of 6 animals (FIG. 11). Within 30-minutes, the unvaccinated animals could be saved by the treatment, showed significantly reduced damage in the striatum, and recovery was observed in 30-days. Vaccinated animals were immune to ET-1 induced MCAO.

NMDA-NR1 r-protein treatment reduced the infarct area in the cortex at several stereotaxic levels in PLGA, Vector and PBS groups (non-vaccinated animals). Brain damage was progressively increased by delay of treatment with NMDA-NR1 r-protein at 60 or 80 minutes after MCA occlusion. Within 30-minutes, animals could be saved by the treatment. They showed significantly reduced damage in striatum and recovered in 80-days. There was a significant linear trend for the degree of increased damage as the time delay increased for treatment with NMDA-NR1 (P=0.02).

Pigs showed neurological deficits indicative of stroke within 5 minutes of injection of ET-1, validating the correct placement of the cannula. The induced stroke resulted in behavior which included circling in the direction contra lateral to the occlusion and failure to extend the contra lateral forepaw. Some pigs showed loss of the righting reflex on the side contra lateral to the MCA occlusion. Histopathology performed on the brains 80 hours after injection of ET-1 showed a pattern of damage similar to that found in other animal species in previous reports (During, et al., 2000. An oral vaccine against NMDAR1 with efficacy in experimental stroke and epilepsy. Science 287:1453-1460; Sharkey et al., 1993. Perivascular microapplication of endothelin-1: a new model of focal cerebral ischaemia in the rat. J Cereb Blood Flow Metab. 13:865-871) which included consistent lesions in the parietal and insular cortex, variable degrees of damage in the frontal cortex, and infarction most often in the dorso-lateral striatum but sometimes extending throughout the corpus striatum.

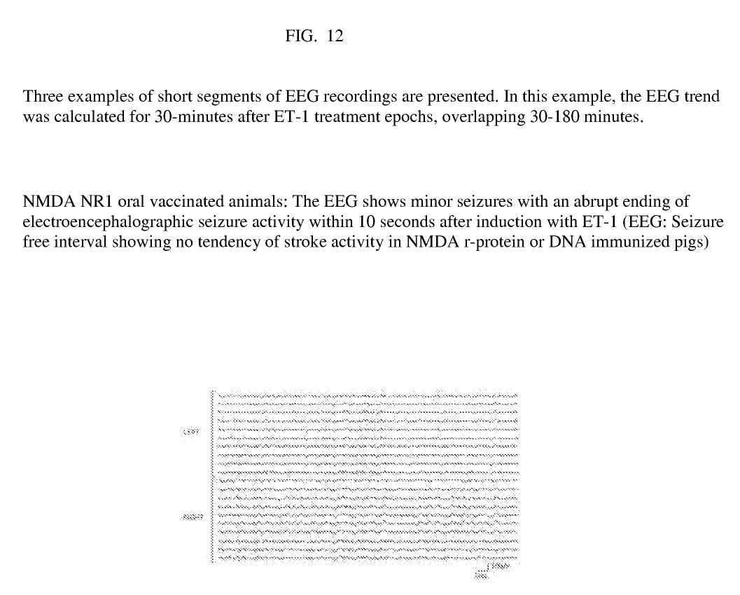

The Electroencephalogram (EEG) in 10 seconds after treating with ET-1: Electroencephalograph (EEG) recordings (FIG. 12) show the ET-1 induced damage in the hippocampus and the neuroprotective effect on stroke status in NMDA-NR1 r-protein or DNA-treated animals compared to controls. The EEG recordings shown in (FIGS. 12 A, B, and C) correspond with the time. ET-1-induced seizures were also elicited in PLGA, pAAV-MCS and PBS-treated animals and TUNEL and clusterin labeling confirmed extensive neuronal damage in the hippocampus. Impairment of motor function or abnormalities in electroencephalographic (EEG) signals was observed. In control animals with traumatic brain injury, stroke or severe hypoxia/ischemia, a type of delayed EEG abnormality or brain seizure evolved that is associated with overt motor convulsions and is therefore clearly identifiable by observers of such animals and could be not be treated with NMDA-NR1 r-protein therapy. Transcranial Arterial Ultrasound Doppler (TCD) and computerized EEG recordings of vaccinated and unvaccinated animals after induction of artificial stroke.

EEG Recording: EEGs were recorded according to the International 10-20 system with Ag/AgCl electrodes, using a bipolar 8-channel subset, using derivations F4-C4, F3-C3, C4-P4, C3-P3, P4-O2, P3-O1, F4-T4, and F3-T3. Impedance was kept <5 kOhm to avoid polarization effects. Recording was performed using a BrainLab EEG recorder. The sampling frequency was set to 250 Hz and filter settings were 0.16 to 70 Hz. EEG recordings lasted more than 30 minutes, whereas for the other clinical indications the total recording time could be limited to 4 minutes at most.

After ET-1 induced artificial stroke in the non-vaccinated animals, a Transcranial Arterial Ultrasound Doppler (TCD) showed diffuse vasospasm with mean flow velocities with an average of 1.7 to 1.81. The flow through the vertebral arteries was 7.1 meters per second. TCD obtained after treatment with NMDA-NR1 protein had flow velocities of 0.49 meters per second with no prolongation of the diastolic flow component. The animals improved dramatically and were able to eat and walk. Among vaccinated animals, TCD readings were same recorded after induction of ET-1 (1.64-1.70) as prior to induction of ET-1. The TCD scores ranged from 0.47-0.49 meters per second and came to normal in 30-minutes without or with ET-1 induction of stroke.

Measurement of Mean Arterial Blood Pressure (MABP) and Rectal and Brain Temperature: Separate groups of animals were anesthetized with halothane (4% for induction; 1-2% for maintenance) in nitrous oxide/oxygen (80/20%; v/v) and placed in a stereotaxic frame. An intravenous catheter was inserted in the femoral artery and connected via a pressure transducer to a Kontron Supermon monitor for measurement of MABP. Rectal temperature was measured by a thermometer inserted into the rectum, which was connected to a thermostatically controlled heating blanket. Brain temperature was measured by a miniature thin film recording probe (Ottosensor, Cleveland, Ohio) inserted into the striatum (AP+1.0 mm; ML . . . 2.0 mm; DV . . . 4 mm below the dura) under stereotaxic guidance. MABP and rectal and brain temperature were recorded for 30 min before induction of focal cerebral ischemia and for 80 min after vessel occlusion.

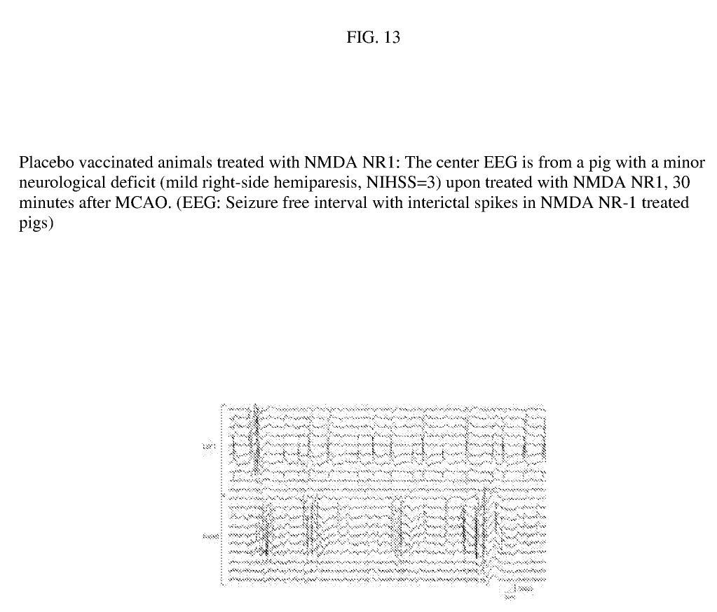

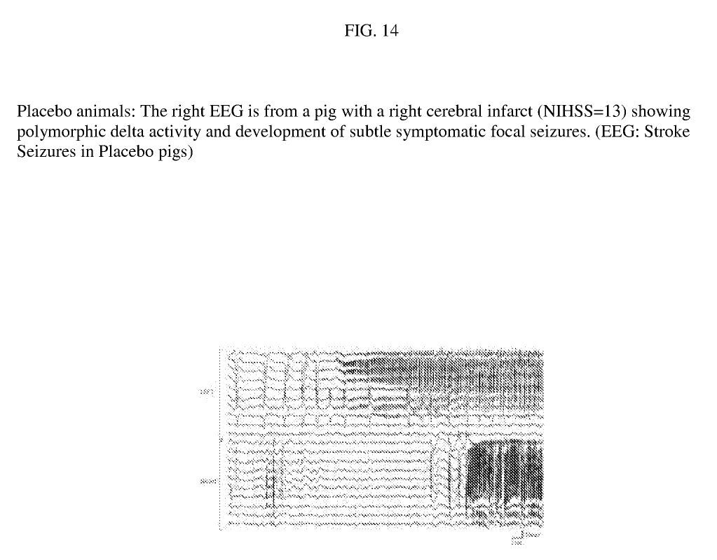

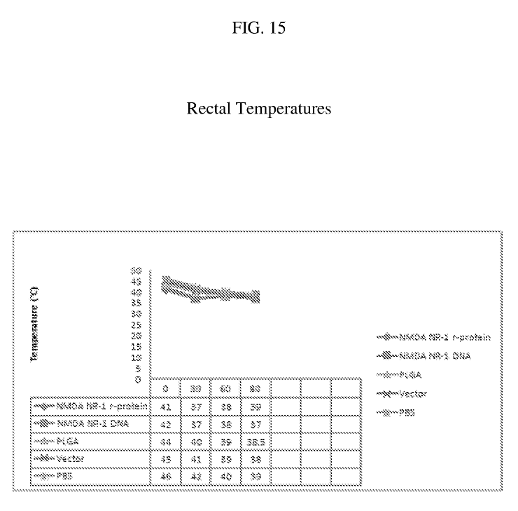

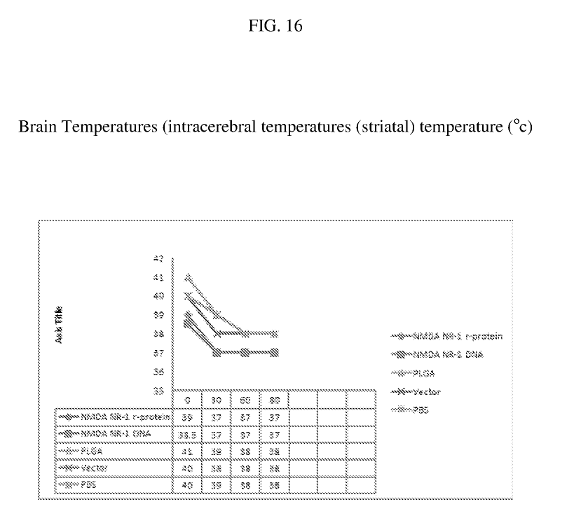

Physiological Variables Mean arterial blood pressure (MABP) was monitored from 0, 30, 60, and 80 min after ET-1 induced MCAO in vaccinated and control animals. Statistically significant effects on MABP were not noted in either vaccinated or unvaccinated groups; rectal and brain temperatures were similarly unaffected after ET-1 induced MCAO in PLGA or pAAV-MCS or PBS-treated pigs (FIGS. 13 and 14).

Rectal temperatures were taken from NR1 vaccinated pigs and from control pigs which were anesthetized prior to induction of stroke with ET-1 and treatment with NR1 protein. At base line (O-min) the rectal temperatures were 39.8 to 42° C. in NR1 vaccinated animals and temperatures dropped to 36-37° C. at 30-minutes and to 37-39° C. at 60-80 minutes. Whereas, in control animals induced with stroke and treated with NR1 protein, at O-minutes the temperatures ranged from 43-47° C., dropped about an average of 5° C. at 30-minutes and dropped about 1° C. at 60-80 minutes (FIG. 13). During the recovery period of 80-hours, the temperatures in all animals came back to the normal temperature average of 40° C. The temperature drop during period of stroke induction might due to anesthesia as reported in some pig studies (Hensel et al., 1995. Oral immunization of pigs with viable or inactivated Actinobacillus pleuropneumoniae serotype 9 induces pulmonary and systemic antibodies and protects against homologous aerosol challenge. Infect Immun. 63:3048-3053).

Brain temperature variation between O-minutes and 80-minutes and further recovery phase of 80-hours, averaged 1.5° C. among the treatment groups of animals (FIG. 14).

Demonstration of NMDA receptor encoding and retrieval of food flavor memory in experimental pigs: Cortical and hippocampal brain regions mediate processes of encoding, storage, and retrieval that specify episodic memory. Representation and retrieval temporally link a series of events, which constitute an episode, mediated primarily by those parts of the cortex that process perceptual and semantic information. It is widely believed that the cortex is mainly involved in coordinating episodic encoding and retrieval and that the medial temporal lobes in the hippocampus store specific aspects of episodic information. One view is that medial temporal lobes store all aspects of episodic memory and retain it for as long as memory lasts. Research has indicated a relationship between stages of neuropathological degeneration and clinical diagnosis suggesting that the entorhinal cortex shows early signs of memory loss linked to the hippocampus (Braak, H. and Braak, E. 1991. Neuropathological staging of Alzheimer-related changes. Acta Neuropathol (Berl). 82:239-259 and Jack et al., 1992. MR-based hippocampal volumetry in the diagnosis of Alzheimer's disease. Neurology 42:183-188). It was reported that the volume of the hippocampi in affected subjects was significantly reduced in comparison to healthy controls.

Further pathological studies have supported these observations with smaller hippocampi and entorhinal cortices among individuals with neurological disorders (Juottonen et al., 1999. Comparative MR analysis of the entorhinal cortex and hippocampus in diagnosing Alzheimer's disease. AJNR Am J. Neuroradiol. 20:139-144). Allocentric place memory may serve to specify the context of events stored in human episodic memory. The same neurobiological substrates relevant to associations of episodic memory in animals could be associated with human episodic memory. An example is the memory of single specific events of places where animals find food with preferred flavor as reported by Tulving (2002. Episodic memory: from mind to brain. Annu Rev Psychol. 53:1-25).

Blockade of remembered events has been reported by several workers (Gaffan, 1991. Spatial organization of episodic memory. Hippocampus 3:262-264; Burgess et al., 2002. The human hippocampus and spatial and episodic memory. Neuron 35:625-641 and Buzsaki et al., 1986. Laminar distribution of hippocampal rhythmic slow activity (RSA) in the behaving rat: current-source density analysis, effects of urethane and atropine. Brain Res 365:125-137). These effects reflect interference with food-finding behavior and demonstrate deficits in episodic-like memory in encoding and retrieval of flavor-place memory. Many animal models emphasize the importance of the hippocampus for rapid encoding and subsequent retrieval of various types of “relational” memory of animals finding buried food. Allocentric place memory requires fast excitatory transmission through hippocampal synapses, essentially mediated by AMPA receptors (Davies S N, Collingridge G L (1989) Role of excitatory amino acid receptors in synaptic transmission in area CA1 of rat hippocampus. Proc R Soc Lond B Biol Sci 236:373-384; Lambert J D C, Jones R S G (1990) A reevaluation of excitatory amino acid-mediated synaptic transmission in rat dentate gyrus. J Neurophysiol 64:119-132). To activate the stored place. Alternatively, it has been suggested that the neural representation of trial-specific places in familiar environments over minutes to hours may not require hippocampal NMDA receptors (Shapiro M L, O'Connor C (1992) N-methyl-D-aspartate receptor antagonist MK-801 and spatial memory representation: working memory is impaired in an unfamiliar environment but not in a familiar environment. Behav Neurosci 106:604-612; Caramanos Z, Shapiro M L (1994) Spatial memory and N-methyl-Daspartate receptor antagonists APV and MK-801: memory impairments depend on familiarity with the environment, drug dose, and training duration. Behav Neurosci 108:30-43; Kesner R P, Rolls E T (2001) Role of long-term synaptic modification in short-term memory. Hippocampus 11:240-250). Studies demonstrated that, analogous to event-place associations in episodic memory, rats could associate, within one trial, a specific food flavor with an allocentrically defined place in an open arena (Bast et al., 2005. Distinct Contributions of Hippocampal NMDA and AMPA Receptors to Encoding and Retrieval of One-Trial Place Memory J. Neurosci. 25:5845-5856). Encoding, but not retrieval, of such flavor-place associations required hippocampal NMDA receptors; retrieval depended on hippocampal AMPA receptors.

The food flavor later serves as a recall cue for the place which is central to episodic memory. We wanted to determine if similar flavor-place associations could be recalled by the pigs vaccinated or treated with NMDA-NR1 and if this differed from behavior of naïve animals. The purpose of these experiments was to see whether the NMDA-NR1 used in vaccination against stroke could affect memory in experimental animals. In addition, we wanted to see if ET-1 induced artificial stroke affected the hippocampus and subsequently affected episodic memory in animals. These experiments are extremely important for functional evaluation of NMDA-NR1 treatment, and its potential for prevention major sequelae of human stroke.

Experimental design: Subjects: Food flavor-place memory recall experiments were conducted prior to necropsy for histopathological examination of brain and other organs. Six pigs from each treatment group and 6 naïve pigs were housed in groups in a pen in a temperature (25 to 28° C.) and humidity (40 to 55%)-controlled room with an artificial light/dark cycle of 11 hr (lights on 6:00 A.M. to 5:00 P.M.). The pigs had access to water and were fed with a restricted diet for 24 hours prior to commencement of the food flavor-place memory recall experiment. Before the start of experiments, all pigs were habituated to handling by the experimenter. All experimental procedures were conducted during the light phase of the cycle.

Food: The favorite food was placed in plastic bowls and buried in circular holes (60 cm diameter, 30 cm deep) in rectangle wood-framed wells so that their edges were plane with the arena surface. The rectangles stood 7.5 cm above the floor (2.0 cm high). Four such food bowls were placed at four corners of a room. The pigs selected in this experiment liked UltraCare™ (Land O'Lakes, Saint Paul, Minn.). This food has a unique, consistent taste and aroma, which pigs recognize from the very start. The UltraCare™ food was adulterated on the top with normal corn feed to 3 cm in two food bowls. The other two food bowls were filled with standard corn pig food. The food bowls were covered with saw dust to reduce the possibility that pigs use food odor to differentiate between UltraCare™ and corn feed in the trials for food flavor-place memory.

Test Room: A rectangular test pen (10×10 m) with white painted wooden walls could be accessed via two yellow painted wooden doors in the two short walls. One door led to the corridor via which the pigs were brought into the test room. A “holding area,” where pigs could be kept in their home pen before and after trials and during delay phases, was located next to this door, separated from the rest of the room by a divider wooden wall. In the center of each wall was a rectangle-shaped entrance with a sliding door and a start box made from clear Plexiglas behind it. A door gave access to a control room with a personal computer and a video recorder, both of which were connected to a wide-angle video camera mounted at the ceiling above the arena. The personal computer was used to monitor trials and to take time measurements, in particular the pig's dig time at different food bowls (measures of one-trial place memory), and for remote control of start-box doors. The video recorder was used to tape the trials.

Food habituation training: During first 5-days of food restriction, pigs were habituated to dig for food in food bowls buried with saw dust in their home pens. In the next 5-days, they were habituated to the test environment and trained to search for food in the arena. Pigs received one daily habituation. In the following habituation trials, one saw dust bowl was half full of UltraCare™, buried on the bottom. The UltraCare™ containing bowl was located immediately in front of the start box opposite to the one from which the pig started the trial. At the end of the 20 day habituation period, all pigs readily searched and dug for food in the arena, indicating completion of habituation to the food trial.

One-trial place memory: Trials were always separated by at least 4 days, with two trials per 10-days. The trials started with the pig placed in a start box. After 10 minutes, the animal was allowed access to the experimental arena. Once the pig had entered the arena, the access door was closed preventing re-entry of the animal to the start box. In the encoding phase, the pig had to search for an open food bowl in one particular location and to dig to retrieve the buried UltraCare™. All other locations were closed and covered with sawdust. After the pig had retrieved the food, it was allowed 60 minutes to eat the reward and then replaced in its pen in the holding area for a delay period until the start of the retrieval phase. The retrieval phase started with the pig being put into a different start box for 30 minutes before the door was opened.

In the retrieval phase, the pig could find food in a food-well in the same location as in the previous location, but four additional food-wells, without reward, were open in four “novel” locations. The pig was returned to its pen 60 minutes after it had retrieved the food. During standard trials, the food reward in the retrieval phase was buried in the same food-well as in the encoding phase. During probe trials, none of the food bowls or food-well in the retrieval phase contained a reward, only normal corn feed, and the pig was left searching and digging in the food-wells for a total of 60 minutes starting from the start box. The purpose of probe trials was to obtain a “food find-time” measure and to make the use of olfactory cues emanating from the food reward impossible.

Measures of one-trial place memory: Measures of one-trial place memory taken during the retrieval phase were (a) the pig's first choice (i.e., in which food-well it dug first), (b) errors (i.e., the total number of food-wells in novel locations in which the pig dug before digging in the food-well in the correct location), and (c) the dig time at correct and novel food-wells during the 60 minute retrieval phase in probe trials. “Digging” was defined as the pig putting its mouth into the bowl buried in sawdust with its snout directed downward, deep in the food-well. In addition to scoring digging based on the video image, the food-wells were checked for holes reflecting that the animal had dug at a given location. The values for the correct choice expected based on chance were 20% of the first choices or 20% of the total dig time at each food-well and an average number of two errors per trial as reported (Clayton et al., 1998. Episodic-like memory during cache recovery by scrub jays. Nature 395:272-274; Clayton and Krebs 1994. One-trial associative memory: comparison of food storing and non-storing species of birds. Anim Learn Behav. 22:366-372 and Clayton et al., 2003. Can animals recall the past and plan for the future? Nat Rev Neurosci 4:685-691).

After habituation and training, the 12 naïve pigs and 6 pigs per treatment had first been subjected to 10 trials of the previously described flavor-place paired-association, as part of ongoing efforts to clarify factors influencing performance in different event-arena protocols.

Results: The purpose of the experiment is to learn if the NMDA-NR1 r-protein/DNA vaccinated and NMDA-NR1 r-protein treated pigs could remember flavor-place associations for a favorite food.

We tested the 6 pre-trained naive pigs and 6 pigs each treatment (NMDA-NR1 r-protein, NMDA-NR1 DNA vaccinated; NMDA-NR1 r-protein treated control animals at various intervals 0, 30, 60 and 80-minutes) for whether they rapidly learned the win stay rule of the flavor-place memory task or not. During the encoding phase, we tested whether they searched the arena for the open food-wells with their heads and retrieved their favorite food at the retrieval phase.

Results are expressed as the percentage of pigs making correct first choices in digging for UltraCare™ food rather than corn feed. The percentages of pigs belonging to a treatment group that showed robust place memory when the delay between encoding and retrieval was extended to 60 minutes is summarized below. The mean number of errors increased or decreased throughout the trials depending on whether not the pig remembered the location of the food which they preferred to eat before enrollment into the experiment. For animals that passed the trial, the percentage of dig time at the food-well in the correct location during the retrieval phase was nearly four times as high as the average at the food-wells in the four locations.

It was demonstrated that 21 animals showed robust place memory when the delay between encoding and retrieval was extended to 60 min and that there was no significant difference in performance between vaccinated, 0 and 30-min treated pigs with NR-1 r-protein and the naive pigs.

Five out of six pigs from NMDA-NR1 r-protein oral vaccinated group, four out of six pigs from the NR1 DNA vaccinated group and 5 out of 6 naïve pigs passed the trial without errors. Among groups treated with NR1 r-protein O-min after ET-1 induced MACO, four of six pigs (PLGA control vaccine), two out of six pigs (vector control vaccine) and five of six pigs (PBS control for vaccine) remembered to dig for and eat UltraCare™.

Whereas, among 30-minute delayed r-NR1 treated pigs 3 out of 6 pigs (PLGA control), 3 out of 6 pigs (vector control) and 4 out of 6 pigs (PBS control) passed the trial. For each group of 60-minute and 80-minute delayed r-NR1 treated pigs 6 about of 6 did not dig for UltraCare™ food.

A clear cut trend of decrease in memory was seen with the delayed treatment of pigs with NR1 r-protein among placebo-vaccinated animals. Most (75%) of the animals vaccinated with NMDA-NR1 r-protein or DNA passed the food memory trial test. Among naïve pigs with no treatment and no ET-1 induced MACO, 5 out of 6 (83%) had not forgotten the flavor-place association for the UltraCare™. This was comparable to vaccinated (75%) and slightly better than 1 and 30-minute treated pigs (58%).

Implications for episodic-like memory by vaccinating and treating with NMDA-NR1: Recently developed episodic-like memory tasks in animals, requiring integrated one-trial memory of events, their places, and their temporal context, depend on the hippocampus (Clayton et al., 2003. Can animals recall the past and plan for the future? Nat Rev Neurosci 4:685-691); Day et al., 2003. Glutamate-receptor-mediated encoding and retrieval of paired-associate learning. Nature 424:205-209; Eacott M J, Norman G (2004) Integrated memory for object, place, and context in rats: a possible model of episodic-like memory? J Neurosci 24:1948-1953; Ergorul C, Eichenbaum H (2004) The hippocampus and memory for “what,” “where,” and “when.” Learn Mem 11:397-405) similar to human episodic memory (Tulving, 2002 Episodic memory: from mind to brain. Annu Rev Psychol 53:1-25). The contributions of hippocampal NMDA receptors to one-trial place memory is probably important for retention of episodic-like memories and may partly explain the requirement of these receptors for one-trial flavor-place memory (Day et al., 2003, see above). However, hippocampal NMDA receptors are critical for rapid encoding of relational memory without a place component Roberts M, Shapiro M L (2002) NMDA receptor antagonists impair memory for nonspatial, socially transmitted food preference. Behav Neurosci 116:1059-1069 and may generally contribute to binding distinct aspects of episodic-like memory, such as flavor and place information, into relational representations (Eichenbaum, 2004. Hippocampus: cognitive processes and neural representations that underlie declarative memory. Neuron 44:109-120).

The hippocampal dorsoventral differentiation could help in dissecting the different hippocampal contributions to episodic-like memory. For example, the dorsal hippocampus, receiving most of the hippocampal visuospatial afferents, may be more important than the ventral hippocampus for encoding one-trial place memory (Bannerman et al., 1995. Distinct components of spatial learning revealed by prior training and NMDA receptor blockade. Nature 378:182-186). In contrast, with substantial olfactory and gustatory input reaching the ventral hippocampus (Petrovich et al., 2001. Combinatorial amygdalar inputs to hippocampal domains and hypothalamic behavior systems. Brain Res Rev 38:247-289). Dorsoventral interactions may be required to bind place and flavor information. In conclusion, the flavor-place association experiments showed extensive memory loss in the unvaccinated, untreated or delayed treatment (60 to 80-min) animals. Moreover, the cortex volume was reduced with these animals compared to the animals vaccinated or treated earlier with NMDA-NR1.

Pre and post-vaccine scenario of NMDA-NR1: Therapeutic effect of NMDA-NR1: Pigs previously immunized with NMDA-NR1 r-protein or DNA and kept for 24 weeks showed minimal stroke related deficits. Neurological scores had improved and were no longer significantly different from those of normal animals and were significantly better than those of non-vaccinated controls after induction of stroke. Humoral immune responses induced by orally delivered PLGA NMDA-NR1 r-protein or DNA microparticles were significantly stronger against ET-1 induced brain damage than those measured in animals immunized with pure PLGA or pAAV-MCS without the NMDA-NR1 insert at the same dose level (P<0.001).

Control pigs exhibited significantly higher neurological deficit scores than NMDA-NR1 vaccinated pigs after induction of artificial stroke. These results confirm that NMDA-NR1 r-protein or DNA vaccines exhibit a powerful neuroprotective action in an experimental swine model for stroke. Additional intravenous studies revealed that NMDA-NR1 r-protein reduces ischemic brain damage in the cortex when administered 0-30 min after MCAO, suggesting that a critical window of opportunity exists with regard to the neuroprotective effect. The ability of intravenous NMDA-NR1 r-protein to reduce cortical brain damage induced by focal cerebral ischemia was consistent with the neuroprotection following oral pretreatment studies using PLGA encapsulated NMDA-NR1 r-protein or DNA. As a noncompetitive NMDAR-antagonist, NMDA-NR1 r-protein administered by an intravenous route reduced ischemic brain damage in the cortex.

An anti-excitotoxic mechanism is likely to underlie the neuroprotective action of NMDA-NR1 in experimental stroke. Pharmacokinetic findings suggest that differences in the time course of excitotoxic and ischemic damage cannot explain the superior efficacy of NMDA-NR1 vaccination. These results suggest that excitotoxins do not exert an effect on the brain that outlives the NMDA-NR1 protein, although there may be an effective concentration of NMDA-NR1 required immediately after the insult to the brain in order to provide an anti-excitotoxic effect. These data appear to indicate that the NMDA-NR1 antibodies block the antagonistic effect of the excitotoxin by binding to the receptor. The ability of the antibodies to enter the brain also suggests that there is a gross perturbation of the blood-brain barrier in the ET-1 model of focal cerebral ischemia.

Physiological data concerning blood pressure, rectal and brain temperature provided no clue to the neuroprotective mechanism of NMDA-NR1 in experimental stroke. MABP was unaffected by NMDA-NR1 pre or post-treatment. Although body and brain temperature influence the severity of brain damage after focal cerebral ischemia (Morikawa et al., 1992. The significance of brain temperature in focal cerebral ischemia: histopathological consequences of middle cerebral artery occlusion in the rat. J Cereb Blood Flow Metab. 12:380-389; Xue et al., 1992. Immediate or delayed mild hypothermia prevents focal cerebral infarction. Brain Res. 587:66-72), these variables were unaltered following ET-1 induced MCAO. These data indicate that neither a direct cardiovascular effect nor a NMDA-NR 1-induced alteration in brain temperature mediates the neuroprotective effect of NR1. The possibility of a direct interaction between anti-NMDA-NR1 antibodies and the endothelin-1 receptors mediating vasoconstriction in this pig model should be considered.

Sequence listing in computer readable form

| NMDA NR1 DNA SEQUENCES | |
|---|---|
| aagtgaccat ggcacatcac caccaccatc acggtggtcc gatgtccact atgcgcctgc | 60 |
| tcacgctcgc cctcctcttt tcttgttctg tagctcgcgc cgcttgtgat ccgaaaatcg | 120 |
| ttaacatcgg cgcagttctg agcactcgta aacatgaaca gatgttccgt gaagccgtaa | 180 |
| accaggccaa caaacgtcac ggttcttgga aaatccagct gaacgcgact agcgtaactc | 240 |
| acaaaccgaa cgctatccag atggcgctct ctgtgtgcga ggatctgatt tcttcccagg | 300 |
| tgtacgcgat cctggtttct catccaccga cgccgaacga ccatttcacc ccaacgccgg | 360 |
| tctcctatac ggcgggcttc taccgcatcc cagtgctcgg tctcaccacc cgtatgtcca | 420 |
| tctactctga caaatctatc catctgtcct ccgtctggtt tgagatgatg cgcgtgtata | 480 |
| gctggaacca cattattctg ctcgtgtccg acgatcacga aggccgtgcg gctcagaaac | 540 |
| gtctcgaaac tctgctcgaa gagcgtgaga gcaaagccga aaaagtcctg cagtttgatc | 600 |
| caggcacgaa aaacgtaact gccctgctca tggaggcaaa agaactggag gcacgcgtaa | 660 |
| ttatcctctc cgcttctgaa gacgatgcag ccactgtcta ccgcgcggcg gcaatgctga | 720 |
| acatgacggg ttctggctac gtgtggctgg taggtgaacg cgaaatctct ggtctccagc | 780 |
| tgattaacgg caaaaacgaa tctgctcata tcagcgacgc cgtcggtgta gtagcacagg | 840 |
| ccgtccacga actcctggag aaagaaaaca ttaccgatcc gccgcgtggt tgcgtcggca | 900 |
| acactaacat ttggaaaacg ggtccgctct ttaaacgtgt cctcatgtct agcaaatacg | 960 |
| cagatggcgt cacgggccgc gttgaattca tcatgaacct ccagaaccgt aaactggtgc | 1020 |
| aggtaggcat ctacaacggc actcatgtca tcccaaacga ccgcaaaatc atctggccgg | 1080 |
| gcggtgaaac cgagaaaccg cgtggctatc agatgtctac ccgcctgaaa attgtaacta | 1140 |
| tccaccagga gccgtttgtg tacgtcaaac caacgctcag cgatggcacc tgtaaagagg | 1200 |
| aatttaccgt caacggcgat ccagtaaaaa aagttatttg cactggtcca aacgatacct | 1260 |
| ctccgggtag cccacgtcat accgttccac agtgttgtta tggtttttgt attgatctcc | 1320 |
| tcatcaaact ggcccgtacc atgaacttta cttacgaagt ccacctcgtg gcagatggta | 1380 |
| aattcggcac ccaggagcgc gttaacaaca gcaacaaaaa agagtggaac ggcatgatgg | 1440 |
| gtgagctgct ctccggccag gctgacatga tcgtcgctcc actgaccatc aacaacgagc | 1500 |
| gcgcacagta cattgagttt tctaaaccgt ttaaatacca gggtctgacc attctcgtga | 1560 |
| aaaaagagat tccgcgttct acgctcgaca gcttcatgca gccgttccag tctactctct | 1620 |
| ggctgctcgt cggtctctcc gtgcacgtcg tcgccgtaat gctgtatctg ctcgatcgct | 1680 |
| tcagcccatt tggtcgcttc aaagtcaact ctgaggagga ggaagaggat gcgctcactc | 1740 |
| tgagcagcgc aatgtggttt agctggggcg tgctcctcaa cagcggtatc ggtgagggtg | 1800 |
| ctccacgtag cttctccgca cgcattctcg gcatggtgtg ggccggtttt gctatgatta | 1860 |
| ttgtagcatc ctacacggcc aacctcgctg cttttctggt cctggatcgt ccagaagagc | 1920 |
| gcatcactgg catcaacgat ccacgtctgc gtaacccatc cgataaattt atttatgcca | 1980 |
| ctgtaaaaca gagcagcgtt gacatttatt tccgtcgcca ggtcgagctg tccacgatgt | 2040 |
| accgtcacat ggaaaaacat aactacgaga gcgcagcaga ggctattcag gcggtccgtg | 2100 |
| ataacaaact ccatgcgttc atttgggatt ctgcggtcct cgaattcgag gcctctcaga | 2160 |
| aatgtgatct ggttaccacg ggcgagctct ttttccgttc cggttttggc attggcatgc | 2220 |
| gcaaagactc cccgtggaaa cagaacgtaa gcctgtccat cctgaaatct cacgaaaacg | 2280 |
| gtttcatgga agacctcgac aaaacctggg ttcgttacca ggaatgcgac tctcgttcta | 2340 |
| acgctccggc aacgctcacc ttcgaaaaca tggctggtgt gttcatgctg gtcgctggcg | 2400 |
| gcatcgtggc aggtatcttt ctcatcttca ttgaaattgc atacaaacgc cataaagacg | 2460 |
| ctcgccgcaa acagatgcag ctcgcctttg cagcagtcaa cgtctggcgc aaaaacctgc | 2520 |
| agcagtacca tccgacggat attaccggtc cgctgaacct gtctgacccg tctgtgagca | 2580 |
| ccgtcgtgta aggatcccaa ttgaggcccc cggaggcgcc cacctgccca gttagcccgg | 2640 |
| ccaaggacac tgatgggtcc tgctgctcgg gaaggcctga gggaagccca cccgccccag | 2700 |
| agactgccca ccctgggcct cccgtccgtc cgcccgccca ccccgctgcc tggcgggcag | 2760 |
| cccctgctgg accaaggtgc ggaccggagc ggctgaggac ggggcagagc tgagtcggct | 2820 |
| gggcagggcc gcagggcgct ccggcagagg cagggccctg gggtctctga gcagtgggga | 2880 |
| gcgggggcta actggcccca ggcggagggg cttggagcag agacggcagc cccatccttc | 2940 |
| ccgcagcacc agcctgagcc acagtggggc ccatggcccc agctggctgg gtcgcccctc | 3000 |
| ctcgggcgcc tgcgctcctc tgcagcctga gctccaccct cccctcttct tgcggcaccg | 3060 |
| cccacccaca ccccgtctgc cccttgaccc cacacgccgg ggctggccct gccctccccc | 3120 |
| acggccgtcc ctgacttccc agctgcagcg cctcccgccg cctcgggccg cct | 3173 |
| NMDA NR1 PROTEIN SEQUENCES | |
| METSERTHRM ETARGLELET HRLEALALEL EPHESERCYS SERVALALAA RGALAALACY | 60 |
| SASPPRLYSI LEVALASNIL EGLYALAVAL LESERTHRAR GLYSHISGLG LNMETPHEAR | 120 |
| GGLALAVALA SNGLNALAAS NLYSARGHIS GLYSERTRPL YSILEGLNLE ASNALATHRS | 180 |
| ERVALTHRHI SLYSPRASNA LAILEGLNME TALALESERV ALCYSGLASP LEILESERSE | 240 |
| RGLNVALTYR ALAILELEVA LSERHISPRP RTHRPRASNA SPHISPHETH RPRTHRPRVA | 300 |
| LSERTYRTHR ALAGLYPHET YRARGILEPR VALLEGLYLE THRTHRARGM ETSERILETY | 360 |
| RSERASPLYS SERILEHISL ESERPHELEA RGTHRVALPR PRTYRSERHI SGLNSERSER | 420 |
| VALTRPPHEG LMETMETARG VALTYRSERT RPASNHISIL EILELELEVA LSERASPASP | 480 |
| HISGLGLYAR GALAALAGLN LYSARGLEGL THRLELEGLG LARGGLSERL YSALAGLLYS | 540 |
| VALLEGLNPH EASPPRGLYT HRLYSASNVA LTHRALALEL EMETGLALAL YSGLLEGLAL | 600 |
| AARGVALILE ILELESERAL ASERGLASPA SPALAALATH RVALTYRARG ALAALAALAM | 660 |
| ETLEASNMET THRGLYSERG LYTYRVALTR PLEVALGLYG LARGGLILES ERGLYASNAL | 720 |
| ALEARGTYRA LAPRASPGLY ILELEGLYLE GLNLEILEAS NGLYLYSASN GLSERALAHI | 780 |
| SILESERASP ALAVALGLYV ALVALALAGL NALAVALHIS GLLELEGLLY SGLASNILET | 840 |
| HRASPPRPRA RGGLYCYSVA LGLYASNTHR ASNILETRPL YSTHRGLYPR LEPHELYSAR | 900 |
| GVALLEMETS ERSERLYSTY RALAASPGLY VALTHRGLYA RGVALGLPHE ASNGLASPGL | 960 |
| YASPARGLYS PHEALAASNT YRSERILEME TASNLEGLNA SNARGLYSLE VALGLNVALG | 1020 |
| LYILETYRAS NGLYTHRHIS VALILEPRAS NASPARGLYS ILEILETRPP RGLYGLYGLT | 1080 |
| HRGLLYSPRA RGGLYTYRGL NMETSERTHR ARGLELYSIL EVALTHRILE HISGLNGLPR | 1140 |
| PHEVALTYRV ALLYSPRTHR LESERASPGL YTHRCYSLYS GLGLPHETHR VALASNGLYA | 1200 |
| SPPRVALLYS LYSVALILEC YSTHRGLYPR ASNASPTHRS ERPRGLYSER PRARGHISTH | 1260 |
| RVALPRGLNC YSCYSTYRGL YPHECYSILE ASPLELEILE LYSLEALAAR GTHRMETASN | 1320 |
| PHETHRTYRG LVALHISLEV ALALAASPGL YLYSPHEGLY THRGLNGLAR GVALASNASN | 1380 |
| SERASNLYSL YSGLTRPASN GLYMETMETG LYGLLELESE RGLYGLNALA ASPMETILEV | 1440 |
| ALALAPRLET HRILEASNAS NGLARGALAG LNTYRILEGL PHESERLYSP RPHELYSTYR | 1500 |
| GLNGLYLETH RILELEVALL YSLYSGLILE PRARGSERTH RLEASPSERP HEMETGLNPR | 1560 |
| PHEGLNSERT HRLETRPLEL EVALGLYLES ERVALHISVA LVALALAVAL METLETYRLE | 1620 |
| LEASPARGPH ESERPRPHEG LYARGPHELY SVALASNSER GLGLGLGLGL ASPALALETH | 1680 |
| RLESERSERA LAMETTRPPH ESERTRPGLY VALLELEASN SERGLYILEG LYGLGLYALA | 1740 |
| PRARGSERPH ESERALAARG ILELEGLYME TVALTRPALA GLYPHEALAM ETILEILEVA | 1800 |
| LALASERTYR THRALAASNL EALAALAPHE LEVALLEASP ARGPRGLGLA RGILETHRGL | 1860 |
| YILEASNASP PRARGLEARG ASNPRSERAS PLYSPHEILE TYRALATHRV ALLYSGLNSE | 1920 |
| RSERVALASP ILETYRPHEA RGARGGLNVA LGLLESERTH RMETTYRARG HISMETGLLY | 1980 |
| SHISASNTYR GLSERALAAL AGLALAILEG LNALAVALAR GASPASNLYS LEHISALAPH | 2040 |
| EILETRPASP SERALAVALL EGLPHEGLAL ASERGLNLYS CYSASPLEVA LTHRTHRGLY | 2100 |
| GLLEPHEPHE ARGSERGLYP HEGLYILEGL YMETARGLYS ASPSERPRTR PLYSGLNASN | 2160 |
| VALSERLESE RILELELYSS ERHISGLASN GLYPHEMETG LASPLEASPL YSTHRTRPVA | 2220 |
| LARGTYRGLN GLCYSASPSE RARGSERASN ALAPRALATH RLETHRPHEG LASNMETALA | 2280 |
| GLYVALPHEM ETLEVALALA GLYGLYILEV ALALAGLYIL EPHELEILEP HEILEGLILE | 2340 |
| ALATYRLYSA RGHISLYSAS PALAARGARG LYSGLNMETG LNLEALAPHE ALAALAVALA | 2400 |
| SNVALTRPAR GLYSASNLEG LNGLNTYRHI SPRTHRASPI LETHRGLYPR LEASNLESER | 2460 |
| ASPPRSERVA LSERTHRVAL VAL | 2483 |

I claim:

1) A method of producing and administering a therapeutic and preventive vaccine for neurodegenerative diseases and neuroendocrine disorders in human, comprising the steps of:
a. construction of N-methyl-D-aspartic acid NR1 (NMDA-NR1) receptor recombinant DNA
b. characterization and expression of the NMDA-NR1 receptor gene
c. encapsulation of NMDA-NR1 recombinant gene with poly-D-L-lactide-co-glycolic acid (PLGA) to produce the vaccine, said vaccine comprising of an antigen capable of eliciting the production of antibodies in the circulatory system of the subject, and said vaccine comprising an insect cell vector vaccine, being selected from the group of a DNA vaccine or peptide vaccine or and crude antigen vaccine or combination thereof and
d. administration of vaccine through oral or intramuscular or intradermal or respiratory route.

2) The vaccine according to claim 1, comprising an antigen selected from the group of neurotransmitters or neuroreceptors or neurotransporters or ion channels, signal transduction molecules, enzymes involved in the synthesis or degradation of neurotransmitters, growth factors, transcription factors and cell surface molecules.

3) The vaccine according to claim 1, comprising NMDA-NR1 r-protein articulated in expression vectors in viruses, bacteria, mammalian cells and stem cells for the production of recombinant protein or any other form or in the form of naked DNA.

4) The vaccine according to claim 1, is administered orally.

5) The vaccine according to claim 1, is administered intramuscularly.

6) The vaccine according to claim 1, is administered intradermally.

7) The vaccine according to claim 1, is administered through respiratory route.

8) The vaccine according to claim 1, providing protection against motor impairment.

9) The vaccine according to claim 1, preventing the onset of the neurodegenerative disease.

10) The vaccine according to claim 1, increasing the survival time of the subject.

11) The vaccine according to claim 1, comprising an antigen which elicits the production of antibodies in the circulatory system of the subject.

12) The vaccine according to claim 1, comprising a viral vector selected from the group consisting of a DNA viral vector.

13) The vaccine according to claim 1, providing immunity against stroke.

14) The vaccine according to claim 1, providing immunity against epilepsy.

15) The vaccine according to claim 1, providing immunity against Alzheimer's disease.

16) The vaccine according to claim 1, providing immunity against Parkinson's disease.

17) The vaccine according to claim 1, providing immunity against dementia.

18) The vaccine according to claim 1, providing immunity against Huntington's disease.

19) The vaccine according to claim 1, providing immunity against amyloid lateral sclerosis.

20) The vaccine according to claim 1, providing immunity against depression.

21) The vaccine according to claim 1, comprising an antigen NMDA-NR1 r-protein.

22) The vaccine according to claim 1, is a genetic vaccine comprising an antigen, NMDA-NR1 r-protein.

23) The vaccine according to claim 1, comprising NMDA-NR1 r-protein opening up of the brain native protein for modulating the nervous system and neurological disorder.

24) The vaccine according to claim 1, comprising NMDA-NR1 r-protein encapsulated with PLGA providing a long term immunity against stroke.

25) The antigen according to claim 1, is NMDA receptor subtype NR1 protein or DNA sequences in the form of alignment presented in this invention.

26) The method according to claim 1, comprising, administration of 100-200 μg of an antigen which is selected from the group comprising neurotransmitters, neuroreceptors, transporters, ion channels, signal transduction molecules or enzymes involved in the synthesis or degradation of neurotransmitters, growth factors and transcription factors and cell surface molecules whereby the antigen binds to and modify the function of the NMDA-NR1 r-protein and thereby ameliorate or prevent the onset of a neurological disorder in the subject.

27) The method according to claim 1, comprising the release of NMDA-NR1 r-protein into the intestine.

28) The method according to claim 1, comprising the expression of the gene NMDA-NR1 receptor in the form of DNA in Insect cells or bacterial cells or mammalian cells or stem cells.

29) The method according to claim 1, comprising the NMDA-NR1 receptor gene expressed as a protein in insect cells or bacterial cells or mammalian cells or stem cells.

30) The vaccine according to claim 1, is an insect cell vector vaccine.

31) The method according to claim 1, comprising poly-d-l-lactide-co-glycolic acid (PLGA) is to increase the residence time of the vaccine.

32) The method according to claim 1, the antibodies pass across the blood-brain barrier into the central nervous system facilitated by injury, disease or excessive neuronal activity.

33) The method according to claim 1, comprising the characterized microspheres for the increased efficiency.

34) The method according to claim 1, comprising NMDA-NR1 r-protein vaccine which developed systemic immunization to generate auto-antibodies in vaccinated humans and animals.

35) The method according to claim 1, comprising optimized polymer encapsulation techniques.

36) The method according to claim 1, representing a unique strategy for the management of stroke injury.


SEQUENCE LISTING (2 sequences)

SEQ ID NO: 1 - DNA, 3173 bp, Homo sapiens
  CDS (9)..(2666)
aagtgaccatggcacatcaccaccaccatcacggtggtccgatgtccactatgcgcctgctcacgctcgccctcctcttttcttgttctgtagctcgcgccgcttgtgatccgaaaatcgttaacatcggcgcagttctgagcactcgtaaacatgaacagatgttccgtgaagccgtaaaccaggccaacaaacgtcacggttcttggaaaatccagctgaacgcgactagcgtaactcacaaaccgaacgctatccagatggcgctctctgtgtgcgaggatctgatttcttcccaggtgtacgcgatcctggtttctcatccaccgacgccgaacgaccatttcaccccaacgccggtctcctatacggcgggcttctaccgcatcccagtgctcggtctcaccacccgtatgtccatctactctgacaaatctatccatctgtcctccgtctggtttgagatgatgcgcgtgtatagctggaaccacattattctgctcgtgtccgacgatcacgaaggccgtgcggctcagaaacgtctcgaaactctgctcgaagagcgtgagagcaaagccgaaaaagtcctgcagtttgatccaggcacgaaaaacgtaactgccctgctcatggaggcaaaagaactggaggcacgcgtaattatcctctccgcttctgaagacgatgcagccactgtctaccgcgcggcggcaatgctgaacatgacgggttctggctacgtgtggctggtaggtgaacgcgaaatctctggtctccagctgattaacggcaaaaacgaatctgctcatatcagcgacgccgtcggtgtagtagcacaggccgtccacgaactcctggagaaagaaaacattaccgatccgccgcgtggttgcgtcggcaacactaacatttggaaaacgggtccgctctttaaacgtgtcctcatgtctagcaaatacgcagatggcgtcacgggccgcgttgaattcatcatgaacctccagaaccgtaaactggtgcaggtaggcatctacaacggcactcatgtcatcccaaacgaccgcaaaatcatctggccgggcggtgaaaccgagaaaccgcgtggctatcagatgtctacccgcctgaaaattgtaactatccaccaggagccgtttgtgtacgtcaaaccaacgctcagcgatggcacctgtaaagaggaatttaccgtcaacggcgatccagtaaaaaaagttatttgcactggtccaaacgatacctctccgggtagcccacgtcataccgttccacagtgttgttatggtttttgtattgatctcctcatcaaactggcccgtaccatgaactttacttacgaagtccacctcgtggcagatggtaaattcggcacccaggagcgcgttaacaacagcaacaaaaaagagtggaacggcatgatgggtgagctgctctccggccaggctgacatgatcgtcgctccactgaccatcaacaacgagcgcgcacagtacattgagttttctaaaccgtttaaataccagggtctgaccattctcgtgaaaaaagagattccgcgttctacgctcgacagcttcatgcagccgttccagtctactctctggctgctcgtcggtctctccgtgcacgtcgtcgccgtaatgctgtatctgctcgatcgcttcagcccatttggtcgcttcaaagtcaactctgaggaggaggaagaggatgcgctcactctgagcagcgcaatgtggtttagctggggcgtgctcctcaacagcggtatcggtgagggtgctccacgtagcttctccgcacgcattctcggcatggtgtgggccggttttgctatgattattgtagcatcctacacggccaacctcgctgcttttctggtcctggatcgtccagaagagcgcatcactggcatcaacgatccacgtctgcgtaacccatccgataaatttatttatgccactgtaaaacagagcagcgttgacatttatttccgtcgccaggtcgagctgtccacgatgtaccgtcacatggaaaaacataactacgagagcgcagcagaggctattcaggcggtccgtgataacaaactccatgcgttcatttgggattctgcggtcctcgaattcgaggcctctcagaaatgtgatctggttaccacgggcgagctctttttccgttccggttttggcattggcatgcgcaaagactccccgtggaaacagaacgtaagcctgtccatcctgaaatctcacgaaaacggtttcatggaagacctcgacaaaacctgggttcgttaccaggaatgcgactctcgttctaacgctccggcaacgctcaccttcgaaaacatggctggtgtgttcatgctggtcgctggcggcatcgtggcaggtatctttctcatcttcattgaaattgcatacaaacgccataaagacgctcgccgcaaacagatgcagctcgcctttgcagcagtcaacgtctggcgcaaaaacctgcagcagtaccatccgacggatattaccggtccgctgaacctgtctgacccgtctgtgagcaccgtcgtgtaaggatcccaattgaggcccccggaggcgcccacctgcccagttagcccggccaaggacactgatgggtcctgctgctcgggaaggcctgagggaagcccacccgccccagagactgcccaccctgggcctcccgtccgtccgcccgcccaccccgctgcctggcgggcagcccctgctggaccaaggtgcggaccggagcggctgaggacggggcagagctgagtcggctgggcagggccgcagggcgctccggcagaggcagggccctggggtctctgagcagtggggagcgggggctaactggccccaggcggaggggcttggagcagagacggcagccccatccttcccgcagcaccagcctgagccacagtggggcccatggccccagctggctgggtcgcccctcctcgggcgcctgcgctcctctgcagcctgagctccaccctcccctcttcttgcggcaccgcccacccacaccccgtctgccccttgaccccacacgccggggctggccctgccctcccccacggccgtccctgacttcccagctgcagcgcctcccgccgcctcgggccgcct

SEQ ID NO: 2 - protein, 860 aa, Homo sapiens
MAHHHHHHGGPMSTMRLLTLALLFSCSVARAACDPKIVNIGAVLSTRKHEQMFREAVNQANKRHGSWKIQLNATSVTHKPNAIQMALSVCEDLISSQVYAILVSHPPTPNDHFTPTPVSYTAGFYRIPVLGLTTRMSIYSDKSIHLSSVWFEMMRVYSWNHIILLVSDDHEGRAAQKRLETLLEERESKAEKVLQFDPGTKNVTALLMEAKELEARVIILSASEDDAATVYRAAAMLNMTGSGYVWLVGEREISGLQLINGKNESAHISDAVGVVAQAVHELLEKENITDPPRGCVGNTNIWKTGPLFKRVLMSSKYADGVTGRVEFIMNLQNRKLVQVGIYNGTHVIPNDRKIIWPGGETEKPRGYQMSTRLKIVTIHQEPFVYVKPTLSDGTCKEEFTVNGDPVKKVICTGPNDTSPGSPRHTVPQCCYGFCIDLLIKLARTMNFTYEVHLVADGKFGTQERVNNSNKKEWNGMMGELLSGQADMIVAPLTINNERAQYIEFSKPFKYQGLTILVKKEIPRSTLDSFMQPFQSTLWLLVGLSVHVVAVMLYLLDRFSPFGRFKVNSEEEEEDALTLSSAMWFSWGVLLNSGIGEGAPRSFSARILGMVWAGFAMIIVASYTANLAAFLVLDRPEERITGINDPRLRNPSDKFIYATVKQSSVDIYFRRQVELSTMYRHMEKHNYESAAEAIQAVRDNKLHAFIWDSAVLEFEASQKCDLVTTGELFFRSGFGIGMRKDSPWKQNVSLSILKSHENGFMEDLDKTWVRYQECDSRSNAPATLTFENMAGVFMLVAGGIVAGIFLIFIEIAYKRHKDARRKQMQLAFAAVNVWRKNLQQYHPTDITGPLNLSDPSVSTVV